(19) United States
(12) Patent Application Publication
(10) Pub. No.: US 2026/0264033 A1
(43) Pub. Date: Sep. 10, 2026

(54) METHOD FOR CONTROLLING A CONTINUOUS GRANULATION AND DRYING PROCESS AS WELL AS INSTALLATION AND SYSTEM THEREFOR

(71) Applicant: Boehringer Ingelheim International GmbH, Ingelheim am Rhein (DE)

(72) Inventors: Moritz Schneider, Ingelheim am Rhein (DE); Victor Nnamdi Emenike, Ingelheim am Rhein (DE); Martin Maus, Ingelheim am Rhein (DE); Judith Stephan (mee Menth), Ingelheim am Rhein (DE)

(21) Appl. No.: 19/136,173

(22) PCT Filed: Dec. 6, 2023
(86) PCT No.: PCT/EP2023/084434

§ 371 Date: Jun. 5, 2025

(30) Foreign Application Priority Data
Dec. 6, 2022 (EP) 22211577.6

(51) Int. Cl. B01J 2/16; G05B 13/02; G05B 13/04

(52) U.S. Cl. CPC B01J 2/16; G05B 13/027; G05B 13/042

(57) ABSTRACT

A method for controlling a continuous granulation and drying process as well as an installation and a system therefor, wherein a model takes into account a combination of granulation with the granulator and subsequent drying with the dryer, and control parameters or forecasted formulation parameters are determined with the model based on state parameters of the installation; and/or wherein the model has a static part with which a base value is or has been determined for the respective control parameter or forecasted formulation parameter with the state parameters, and the model has a dynamic part with which the base value is optimized using a forecast.

### BACKGROUND OF THE INVENTION

### Field of the Invention

The present invention relates to a method for controlling an installation as well as an installation and a system with the installation.

### Description of Related Art

The background of the present invention primarily concerns the production of drug administration forms, specifically tablets, capsules, or granules. In principle, however, the invention can also be used in other technical fields, in particular if a formulation with a specified/preset or presettable attribute relating to particles of the formulation such as a specified/preset particle size distribution and/or moisture is to be produced. This is the case, for example, if the formulation is to be tableted, which is indeed particularly preferably the case in the pharmaceutical field, but is in principle also possible in connection with cleaning agents, foods or the like.

The present invention relates in particular to a method as well as an installation for the preferably continuous production of a formulation from a feedstock. Very particularly preferably, the installation is controlled with a proposed method in such a way and/or the installation is configured in such a way that a formulation with a specified/preset or presettable property relating to particles of the formulation, such as homogeneity and/or particle distribution, and, preferably, a specified/preset or presettable (relative) moisture is produced from the feedstock.

The combination of a granulator and a dryer has proven to be advantageous for the production of the formulation from the feedstock. Thus, an intermediate product (granulate) with a specified/preset and/or presettable particle distribution can initially be achieved with the granulator starting from the feedstock, while the intermediate product can subsequently be conditioned to a specified/preset or presettable (relative) moisture by means of the dryer.

One difficulty here consists in the fact that the varying properties of the intermediate product have effects on the drying and thus on the formulation.

In principle, fluidized bed granulators are known. In the solution disclosed there, a specific particle distribution and conditioning with regard to a relative moisture are effected in the same step. Solutions based on fluidized bed granulators, however, have disadvantages with regard to the reliable generation of an exact particle size distribution and moisture and, in addition, are suitable only for specific feedstocks.

In comparison thereto, the use of a granulator followed by the use of a dryer realized separately therefrom has proven to be more advantageous, since here the components can also be used separately from one another and, if appropriate, a more exact particle size distribution and relative moisture can be achieved, preferably at least substantially independently of one another. The proposed method can, however, in principle also be advantageous for controlling fluidized bed granulators.

Combinations of a granulator and a dryer are also known in principle, firstly in batch operation. In a batch process, a batch of the feedstock is subjected to a first production step and subsequently the entire batch is subjected to another, second production step before the result, i.e. the formulation, results from the production process.

In contrast thereto, in a continuous production process, which preferably forms the basis of the present invention, it is the case that, after a start phase, feedstock is supplied at the same time, while previously supplied feedstock is subjected to the production process and again previously supplied feedstock, which has already been completely subjected to the production process, is removed. Thus, in a continuous process, at the same time feedstock is supplied and the result is removed in the form of the formulation.

### SUMMARY OF THE INVENTION

The present invention preferably relates to a continuous production of the formulation and/or to a continuous method and the installation therefor and/or to the system with the installation, preferably in contrast to a batch process. Advantages of a continuous process are:
simple scalability (scaling possible according to time and throughput)
small space requirement of the installation
shorter downtimes of the installation in comparison to batch installations
higher degree of automation possible
higher product quality

It is the object of the present invention, against this background, to provide a method as well as an installation and a system, by means of which the process for producing the formulation from the feedstock can be improved with regard to a reliable and unchanging realization of attributes such as, in particular, a particle size distribution and moisture.

This object is achieved by a method, an installation or a system as described herein.

The present invention relates on the one hand to a method for controlling an installation for producing a formulation from a feedstock, wherein the production comprises a processing of the feedstock with a granulator and a drying of an intermediate product produced from the feedstock with the granulator by means of a dryer.

In a first variant of the present invention, control parameters of the installation are determined based on a model. Here, the model takes into account preset target formulation parameters, which represent desired properties of the formulation produced or to be produced.

The desired properties of the formulation produced or to be produced is in particular an intended grain property and moisture.

Here, variably presettable target formulation parameters, with which the model determines the control parameters, can be passed to the model. However, the target formulation parameters can alternatively or additionally be taken into account or have been taken into account when forming the model.

The control parameters are or represent manipulated variables for controlling actuators of the installation.

State parameters of the installation are determined and processed by the model. For this purpose, they are preferably passed to the model and used by the latter for determining the control parameters.

The state parameters each represent a state of the installation which influences the production and are preferably sensor values.

Furthermore, feedstock parameters are taken into account by the model. In particular, variably presettable feedstock parameters, with which the model determines the control parameters, are passed to the model. However, the feedstock parameters can alternatively or additionally be at least partly taken into account or have been taken into account when forming the model.

The feedstock parameters represent an attribute of the feedstock, in particular a moisture and/or a grain property.

In one aspect, the dryer is coupled to the granulator in such a way that the intermediate product is automatically conveyed without interruption from the granulator into the dryer. According to the proposal, the model takes into account the continuously running combination of granulation with the granulator and subsequent drying with the dryer.

In a second aspect which can be combined with the first aspect, the model has a static part, with which a base value is determined for the respective control parameter, and a dynamic part, with which the base value is optimized by means of a forecast.

In a second variant of the present invention, actual formulation parameters are forecast based on a (the same or a different) model.

Here, the model takes into account the control parameters which are preset or presettable in this aspect. Preferably, variably presettable control parameters, with which the model forecasts the actual formulation parameters, are passed to the model. However, the control parameters can alternatively or additionally be at least partly taken into account or have been taken into account when forming the model.

The actual formulation parameters represent actual properties of the formulation produced or to be produced, in particular an actual grain property and moisture.

As already in the first variant, the state parameters are determined and processed by the model and the feedstock parameters are taken into account by the model.

Furthermore, as already in the previous variant, in one aspect, the dryer is coupled to the granulator in such a way that the intermediate product is automatically conveyed without interruption from the granulator into the dryer. According to the proposal, the model takes into account the continuously running combination of granulation with the granulator and subsequent drying with the dryer.

In a second aspect which can be combined with the first aspect, the model also has, in the second variant, a static part, with which a base value is determined for the respective control parameter, and a dynamic part, with which the base value is optimized by means of a forecast.

In a proposed method, for controlling the installation for the production of the formulation from a feedstock or for supporting the control, feedstock parameters are therefore first preset or taken into account, which represent a state of the feedstock, in particular a moisture and/or a particle property such as a particle size distribution.

A feedstock in the sense of the present invention is preferably a granulatable substance, i.e., a substance which can be processed by a granulation process to form a granulate. The feedstock is very particularly preferably a powder or granulate, the particle properties of which can be changed by a granulation process.

Furthermore, the feedstock is preferably a substance mixture, i.e. an at least substantially homogeneous mixture of different, preferably solid, components. These components can have an active substance, in particular a pharmacologically or otherwise active substance, a filler and/or a disintegrant. In particular, the feedstock is an at least substantially homogeneous powder mixture.

In the production of the formulation from the feedstock, material parameters of the feedstock, passing through the installation, via its intermediate product up to the end product (formulation) are preferably not determined by sampling and analysis away from the installation. Instead, measurable parameters are used exclusively in ongoing, continuous operation.

Either no material parameters are determined at all or at most in-line measurable material parameters, for example measurement values from contactless measuring methods, a reflection and/or transmission measurement, in particular with infrared radiation, for example as an indicator for the material moisture. The measurement of particle size distributions in the production process is preferably dispensed with, at least for the intermediate product.

For the proposed control, control parameters of the installation can be determined, which are or represent manipulated variables for controlling actuators of the installation. The installation can thus be controlled with these control parameters. This is preferably done by controlling different actuators of the installation with the control parameters, so that they influence the feedstock and/or the intermediate product.

Alternatively, or additionally, for supporting the control, actual formulation parameters are forecast, which represent properties of the produced formulation under specified/preset boundary conditions. This can be done on the basis of the preset or presettable control parameters.

In other words, actual formulation parameters can be forecast and preferably output with preferably manual provision or input of the control parameters. This allows a user a—again preferably manual—comparison with target formulation parameters and a specification/provision of varied control parameters, in order to match the forecasted actual formulation parameters to the target formulation parameters. The varied control parameters are then preferably used as the basis for the control of the installation.

Preferably, at least one granulator drive and a supply for a desiccant, in particular (conditioned) air, are controlled with the control parameters. In addition, a supply device for the feedstock, an injection for liquid during granulation, one or more temperature control device(s) of the granulator, a conveying device for the desiccant for setting a desiccant volume flow and/or a temperature control device for temperature control of the desiccant can be controlled with the control parameters.

For the control of the installation and/or for supporting the control, state parameters of the installation, in particular one or more sensor values, are determined, which preferably each represent a state of the installation which influences the process for producing the formulation from the feedstock. These include, in particular, temperatures and/or pressures and/or pressure differences and/or torques and/or volume flows. However, other parameters and/or sensor values which describe a state of the installation are also conceivable.

However, the state parameters preferably do not describe, or at least do not directly describe, material properties of the feedstock or of an intermediate product (granulate) or end product (formulation) formed therefrom.

In any case, it is preferred that, in the continuously running process of granulation and drying, no particle size distribution, no size, no shape, no density and/or no active ingredient content of the feedstock or intermediate product formed therefrom is/are determined. In this respect, the present invention adopts a completely different approach compared to the prior art. By contrast, properties of the feedstock can be determined in advance and properties of the end product, i.e. of the formulation, can be determined for verification after completion and/or for forming a model.

Target formulation parameters are preferably preset for the control, which represent the desired properties of the formulation produced or to be produced, in particular one or more physical properties such as a particle size, particle size distribution, particle shape or density and/or a moisture of the formulation.

Furthermore, for model formation and/or verification, preferably by characterization of the formulation, actual formulation parameters are determined, which represent the actual properties of the formulation produced or to be produced, in particular one or more physical properties such as a particle size, particle size distribution, particle shape or density and/or a moisture of the formulation.

Finally, it is provided that the control parameters are determined based on the model. The installation can then be controlled with the control parameters, which are preferably determined by processing the state parameters with the model, in the process for producing the formulation.

The (measured) actual formulation parameters are preferably used to derive and/or define the model. However, the (measured) actual formulation parameters are preferably not used as the basis for the control of the installation in the ongoing process.

The control parameters are therefore preferably not determined by processing the (measured) actual formulation parameters and/or derived therefrom. This is because it has surprisingly been found that a derivation of the control parameters from (measured) actual formulation parameters starts too late. If (measured) actual formulation parameters deviate from the target formulation parameters in the ongoing process, considerable scrap is already preprogrammed. However, the object of the invention is to avoid such scrap. For this purpose, it is rather preferred to control the installation (at least substantially) independently of the (measured) actual formulation parameters or for the installation to be configured therefor.

The (measured) actual formulation parameters are preferably used to determine the model and/or to derive a scheme in the form of the model, with which the control parameters are derived from the state parameters of the installation during the process.

The model preferably has a machine-learning-based structure, in particular a neural network.

As already mentioned, in a first aspect of the present invention, the installation has a granulator for processing the feedstock to form an intermediate product and a dryer, which is coupled to the granulator in such a way that the intermediate product is automatically conveyed without interruption from the granulator into the dryer.

Here, the model takes into account, in particular describes, the combination of granulation with the granulator and the subsequent drying with the dryer. With the model, the control parameters, in particular base values, of the control parameters can be determined based on the state parameters of the installation, and preferably the feedstock parameters.

Alternatively or additionally, forecasted (thus not measured) actual formulation parameters are determined based on the model. This may be a different model than for determining the control parameters. The forecasted actual formulation parameters can be output, in order to allow a (manual) comparison with target formulation parameters and, if appropriate, a (manual or automatic) adjustment of the control parameters. In this case, the control parameters are preferably preset for the model.

Optionally, one or more in-line measurable properties of the intermediate product, in particular one or more parameters optically measurable on the intermediate product, can be additionally taken into account. Measurement values whose determination requires sampling, analysis separate from the installation or interruption of the production process, however, are preferably avoided.

It is therefore not excluded that, in addition to state parameters of the installation, certain (physical) in-line measurable properties of the feedstock and/or of the intermediate product and/or of the formulation (actual formulation parameters) is/are determined or used for closed-loop control and/or as an input variable of the model, for example a moisture and/or particle size distribution of the formulation 7 or variables determined therewith, in particular if an in-line measurement is possible for determination without interruption of the continuous granulation and drying.

Preferably, a measurement of a particle size distribution at most or only of the formulation is provided here, but not of the intermediate product. Accordingly, the installation can have a sensor for determining an attribute describing a particle of the formulation such as a particle size distribution, but preferably only at or downstream of the formulation outlet for dispensing the formulation after drying of the intermediate product. The sensor provided as part of the installation or of a system with the installation is in particular an in-line probe with local filter anemometry for particle size measurement. However, other principles are also possible here.

A moisture is preferably determined from the formulation, but alternatively or additionally can also be determined from the intermediate product. One or more sensors can be provided for this purpose. In particular, this is an optical sensor, particularly preferably an infrared radiation-based sensor. In this context, sensors based on near-infrared radiation, in particular from the NIR-2 spectrum of 860 to 1040 nm wavelength, have proven to be particularly advantageous.

It has proven to be particularly advantageous independently of and synergistically with the model formation of the combination of granulator and dryer if the model has a static part, with which a base value is determined for the respective control parameter and/or forecasted actual formulation parameter from the feedstock parameters and the state parameters, and the model furthermore has a dynamic part, with which the base value is optimized or can be optimized by means of a forecast.

The static part of the model can be defined by determining model parameters which are determined, in particular measured, corresponding to one another during a plurality of respectively stationary (steady state, at least substantially static) states of the installation.

With the static part of the model, base values of the control parameters can be determined from the state parameters and/or the actual formulation parameters can be forecasted. The static part of the model is preferably provided invariably.

With the dynamic part of the model, the behavior of the model can be continuously adjusted. For example, the behavior of an installation changes over time on account of wear, material expansion, aging, or the like. Instead of taking into account such or similar transient effects directly by a change (in particular scaling and/or correction by addition/subtraction of correction terms) of values of the control parameters or forecasted actual formulation parameters, the model is adjusted according to the proposal such that the model generates corrected control parameters and/or actual formulation parameters from the state parameters.

One or more, if appropriate different, of the parameters (for example the state parameters) are processed by the model. Here, it can be provided that these parameters form input values of the model, thus in particular of the static part of the model and/or of the dynamic part of the model.

The model, in particular the static part thereof, can have an artificial neural network. The parameters can be passed to nodes of an input layer of the artificial neural network and the artificial neural network can use these to generate values at the nodes of an output layer of the artificial neural network.

The values generated at the nodes of the neural network of the static part of the model in the output layer can be optimized with the dynamic part of the model, for example by processing taking into account the same and/or other parameters.

A further aspect of the present invention, which can also be realized independently, relates to a computer program product or computer-readable storage medium, comprising commands which, when the program is executed by a computer, cause the latter to carry out the method according to one of the described aspects.

A further aspect of the present invention, which can also be realized independently, relates to the installation for producing the formulation from the feedstock, wherein the production comprises a processing of the feedstock with a granulator and a drying of an intermediate product produced with the granulator by means of a dryer.

The installation has the sensors for detecting state parameters of the installation, which each represent a state of the installation which influences the production. The installation has the actuators for acting directly or indirectly on the feedstock. Furthermore, the installation has a control device for controlling the actuators with the control parameters, wherein target formulation parameters are preset or presettable for the control device, and the model, on the basis of which the control parameters are determinable.

The granulator is coupled to the dryer in such a way that the intermediate product is automatically conveyed without interruption from the granulator into the dryer, wherein the model takes into account the combination of granulation with the granulator and the continuous subsequent drying with the dryer, and the installation is configured such that the control device determines the control parameters with the model based on the state parameters of the installation.

Alternatively or additionally, the model has a static part, wherein the control device is configured to determine a base value with the static part for the respective control parameter from the feedstock parameters and the state parameters, and the model has a dynamic part, wherein the control device is configured to optimize the base value with the dynamic part by means of a forecast. The installation is then preferably controlled with the resulting, optimized base value, and/or the installation is controllable therewith.

A further aspect of the present invention, which can also be realized independently, relates to a system comprising the proposed installation and a device for forming the feedstock from a plurality of components, preferably powders, preferably by sieving, and/or a device for further processing, preferably tableting, of the formulation.

A formulation in the sense of the present invention is a substance which has passed through the installation and has been changed thereby with regard to its physical properties. The formulation is thus preferably the product of the combination of granulation and drying. A subsequent further processing of the formulation, which is preferably present as (dried and/or preferably conditioned with regard to its (relative) moisture) granulate, for example by tableting, is not excluded.

A feedstock in the sense of the present invention is preferably a substance which is supplied to the installation and/or to the granulator in order to change its physical properties. The feedstock is preferably an active ingredient-filler mixture. However, this is not mandatory. The feedstock can already be preprocessed, for example by at least substantially homogeneous mixing of a powder with another powder or another substance, one of which can be or have an active substance. An active substance is preferably a pharmacologically active substance.

A granulator in the sense of the present invention is preferably a device which mechanically processes the feedstock in order to change its physical properties. Particularly preferably, the granulator converts the feedstock into a granulate, in particular a coarse (granular) powder. For this purpose, the feedstock can be supplied to the granulator as a powder in order to process it into a coarser-grained or finer-grained powder. The intermediate product is preferably a solid.

Preferably, the granulator conveys the feedstock while it acts on it. The granulator preferably produces pressure and/or friction in the feedstock, preferably with temperature control/heating and/or supply of moisture. Particularly preferably, the granulator changes the granularity, grain size or particle size distribution of the feedstock. In the present invention, the granulate is an intermediate product which is further processed in the following.

The granulator can have an extruder or be formed by an extruder. In particular, this is a screw extruder, preferably a twin-screw extruder, or the granulator has or is similar to such a screw extruder. However, other solutions are also conceivable here.

The granulator is preferably a screw granulator, for example a twin-screw granulator. In screw granulators, the feedstock is conveyed and processed by means of a screw shaft rotating about an axis of rotation. For this purpose, the screw shaft can have screw flights of different pitch and/or processing structures. In a twin-screw granulator, two screw shafts are provided which are arranged in parallel and/or engage in one another and effect the conveying and processing. In principle, other concepts can also be used here. The conveying preferably results in an extrusion. The screws are therefore or form one or more extruders and/or effect an extrusion of the feedstock.

The granulator is preferably a granulator for wet granulation. For this purpose, the granulator can have an injection of liquid for moistening the feedstock, whereupon the moistened feedstock is processed by means of the granulator to form the intermediate product. The granulator can have an opening, in particular a nozzle, and/or a valve for adding the liquid to the feedstock, in particular to add water, ethanol, isopropanol and/or a mixture thereof, for the purpose of (temporarily) increasing the moisture.

A dryer in the sense of the present invention is a device for reducing a (relative) moisture and/or a water content of a substance, in the present case for reducing a (relative) moisture and/or the water content of the feedstock processed with the granulator into an intermediate product. The dryer is preferably a device which withdraws water from the substance/intermediate product. This is preferably a device which brings the substance/the intermediate product into contact with a desiccant which withdraws water from the substance.

Preferably, the desiccant is air or another gas with a relative humidity which allows the absorption of water from the substance. Preferably, the desiccant is temperature-controlled, in particular to a temperature above the ambient temperature. The desiccant is thus in particular preconditioned, warm and/or dried air, also called process air. In principle, however, it can also be other, in particular inert gases or other, preferably gaseous, desiccants.

The dryer is preferably a fluidized-bed dryer. A fluidized-bed dryer in the sense of the present invention is a device which produces a cushion of air or gas (process air) for a substance to be dried, in this case for the intermediate product/granulate. In this context, the air or the gas is the preferably gaseous desiccant.

The preferably gaseous desiccant is supplied to a bed of the dryer from the intermediate product/granulate, preferably through a perforated distributor plate. The preferably gaseous desiccant flows at a speed through the bed, so that the particles of the intermediate product/granulate are kept in a fluidized state despite their weight. The fluidized particles of the intermediate product/granulate form the fluidized bed. Here, they are dried by preferably gaseous desiccants.

Within the fluidized bed, bubbles can form and collapse again in order to promote an intensive particle movement. In this state, the solids behave like a free-flowing, boiling liquid. Very high heat and mass transfer rates are the result of the close contact between the individual particles and the preferably gaseous desiccant. In principle, however, other dryer concepts can also be used, even if fluidized-bed dryers have proven to be particularly advantageous in the context of the present invention.

Sensors for detecting state parameters of the installation, which each represent a state of the installation which influences the production, in the sense of the present invention are preferably sensors which characterize a state of devices of the installation, i.e. are oriented and configured for measuring one or more state parameters.

State parameters in the sense of the present invention can be or represent one or more of the following attributes:

Moments/torques and/or speeds of drives, tools and/or conveying devices (in particular of propellers, turbines, screws/extruders) or parameters corresponding thereto, such as current consumption and/or rotational speeds

Mass flow rates (of desiccant/process gas, feedstock, aggregate/granulating liquid, intermediate product, exhaust gas, end product/formulation) or parameters corresponding thereto, which characterize, for example, positions of valves, flaps, rotor rotational speeds, pressure differences or the like

Temperatures of parts of the installation, which are preferably in direct or indirect contact with the feedstock being processed, with the aggregate/granulating liquid or with the desiccant, of tools for processing the feedstock or the intermediate product, of devices for adding aggregate or introducing desiccant

State parameters preferably do not characterize (directly) any (physical or chemical) property of the feedstock or of an intermediate product or end product formed therefrom and/or of the formulation. Therefore, in particular, no measured variable of the feedstock or of an intermediate product or end product (formulation) formed therefrom, which characterize a chemical composition or the granularity, is considered to be a state parameter of the installation.

In principle, a distinction can be made between two groups of state parameters.

A first group includes state parameters which are at least substantially independent of the feedstock, in particular in that they are set directly by an actuator of the installation. Henceforth, these are also referred to as presettable and/or “feedstock-uninfluenced state parameters”. Examples for this are presettable temperatures or rotational speeds. They can be used in particular for actuator control.

State parameters of a second group which are influenced by interaction with the feedstock or with the intermediate product formed therewith can be distinguished from the “feedstock-uninfluenced state parameters”. Henceforth, these are referred to as “feedstock-influenced state parameters”. Examples for this are a torque occurring as a function of a consistency of the feedstock or a (relative) humidity of an exhaust air moistened by the drying process.

One or more feedstock-influenced state parameters are preferably used as input variables for the model. The state parameters which are processed by the model and/or passed to the model for this purpose and are used by the latter for determining the control parameters or for forecasting the actual formulation parameters are thus preferably feedstock-influenced state parameters.

The state parameters which are processed by the model and/or passed to the model for this purpose and are used by the latter for determining the control parameters or for forecasting the actual formulation parameters are preferably at least one feedstock-influenced state parameter of the granulator, further preferably at least one feedstock-influenced state parameter respectively of the granulator and of the dryer, in particular at least two feedstock-influenced state parameters of the granulator and at least one, preferably at least two, feedstock-influenced state parameters of the dryer.

Preferably, one or more of the feedstock-uninfluenced parameters are used for closed-loop controlling the corresponding actuator. Alternatively or additionally, one or more feedstock-influenced state parameters are used for determining the control parameter or parameters, preferably by means of the model. The control parameters can be target specifications for closed-loop controlling the actuator or actuators, on the basis of which the actuators are then closed-loop controlled.

An in-line measurable property in the sense of the present invention is preferably property that can be measured incidentally in the uninterrupted, continuous manufacturing process.

Actuators for acting on the feedstock in the sense of the present invention are preferably drives of tools and/or conveying devices. These may be motors which are used, for example, for driving fans, turbines, conveyor belts and/or screws, or also temperature control devices, heater or cooler (for example a cooling water conveying device) for temperature control of feedstock-carrying housing parts of the granulator. A temperature control device, in particular a heater or cooler such as a heating register, for temperature control of the process gas may also be an actuator, since it acts on the feedstock indirectly via the temperature of the desiccant/process gas.

A control device for controlling the actuators in the sense of the present invention is preferably an electronic component for influencing the operation of the actuators, for controlling motor rotational speeds (of the screw drive and/or of the fan motor(s)) and/or for open-loop controlling or closed-loop controlling temperature control devices and/or heaters of the granulator and/or for the desiccant.

Control parameters which are or represent manipulated variables for controlling the actuators in the sense of the present invention are preferably values as specifications for the operation of the actuators, in particular one or more specified/preset motor rotational speeds (of the screw drive(s) and/or of the fan motor(s)) or current consumption corresponding thereto and/or specifications for open-loop controlling or closed-loop controlling temperature control devices and/or heaters of the granulator and/or for the desiccant or the like.

Feedstock parameters in the sense of the present invention are preferably parameters which characterize preferably physical properties of the feedstock, such as a granularity, particle size, particle size distribution, (relative) moisture and/or temperature of the feedstock.

A state of the feedstock in the sense of the present invention is preferably determined at least by its moisture and/or a particle size distribution, optionally supplemented by further properties or replaced by corresponding information which allows conclusions to be drawn directly or indirectly about the moisture and/or a particle size distribution or is derived therefrom.

Target formulation parameters in the sense of the present invention are preferably parameters which represent the desired properties of the formulation produced or to be produced, in particular a granularity and/or particle size distribution and a moisture, optionally supplemented by further properties or replaced by corresponding information which allows conclusions to be drawn directly or indirectly about the moisture and/or a particle size distribution or is derived therefrom.

Actual formulation parameters in the sense of the present invention are parameters measured on the formulation, which represent the properties of the formulation produced or to be produced, in particular a particle size, particle size distribution and a moisture, optionally supplemented by further properties or replaced by corresponding information which allows conclusions to be drawn directly or indirectly about the moisture and/or a particle size distribution or is derived therefrom.

Actual formulation parameters can be forecasted as an alternative to the measurement, but are then referred to in the following as forecasted actual formulation parameters.

A model in the sense of the present invention is preferably a (abstracted) representation preferably restricted to essential properties. A model here is preferably a model of the installation and represents-preferably mathematically-properties of the installation directly or indirectly by its effects on the feedstock and/or the intermediate product. For this purpose, the model can have or be formed by a mathematical description of the granulator, of the dryer and/or of their effects on the feedstock and/or the intermediate product. The model preferably makes it possible, starting from state parameters of the installation and, if appropriate, feedstock parameters, to determine control parameters and/or to forecast formulation parameters.

A coupling of the granulator to the dryer in the sense of the present invention is preferably a device for transferring the intermediate product into the dryer and can have a conveying device for this transfer, such as a chute, a conveyor belt, a screw or the like. The coupling preferably has the effect that the intermediate product is automatically conveyed continuously, without interruption and/or “in-line” from the granulator into the dryer. For this purpose, the granulator can also have an outlet which opens directly into the dryer. Here, the coupling is therefore effected by the granulator and/or by the conveying action of the granulator.

Basic values of the control parameters in the sense of the present invention are preferably control parameters which serve as basic settings and are preferably determined or specified/preset independently of measured properties of the end product (the formulation). Basic values of forecasted actual formulation parameters are starting points for the forecast.

A static part of the model in the sense of the present invention is preferably a part of the model based on empirical values, with which basic values can be determined and/or forecasted.

A dynamic part of the model in the sense of the present invention is preferably a part of the model which can be changed as a function of state parameters of the installation dynamically, i.e. in ongoing operation of the installation during the processing of the feedstock to the intermediate product and end product, of the formulation, in order to adjust the model accordingly to any (future) changes in states of the installation or of the process, preferably so that the model represents the behavior of the installation and/or the process for producing the formulation from the feedstock preferably with sufficient accuracy. The dynamic part of the model preferably makes it possible to optimize the basic value or values by means of a forecast. Past developments can be taken into account for this purpose.

Further aspects, advantages and properties of the present invention will be apparent from the following descriptions of a preferred exemplary embodiment with reference to the accompanying drawings.

### BRIEF DESCRIPTION OF THE DRAWINGS

FIG. 1 shows a schematic section of the proposed installation;

FIG. 2 shows a simplified, block diagram-like view of control-relevant components of the installation according to FIG. 1;

FIG. 3 shows a simplified, schematic view of an artificial neural network;

FIG. 4 shows a schematic diagram of past and forecasted time courses;

FIG. 5 shows a schematic diagram of the result of a control with constant control parameters over time;

FIG. 6 shows a schematic diagram of forecasts offset in time with respect to one another;

FIG. 7 shows a schematic diagram of process properties over time;

FIG. 8 shows a schematic flow diagram;

FIG. 9 shows an installation embedded in a system.

### DETAILED DESCRIPTION OF THE INVENTION

In the drawings, the same reference signs are used for identical or similar parts, wherein the same or similar properties and advantages can be achieved, even if a repeated description is dispensed with for reasons of clarity.

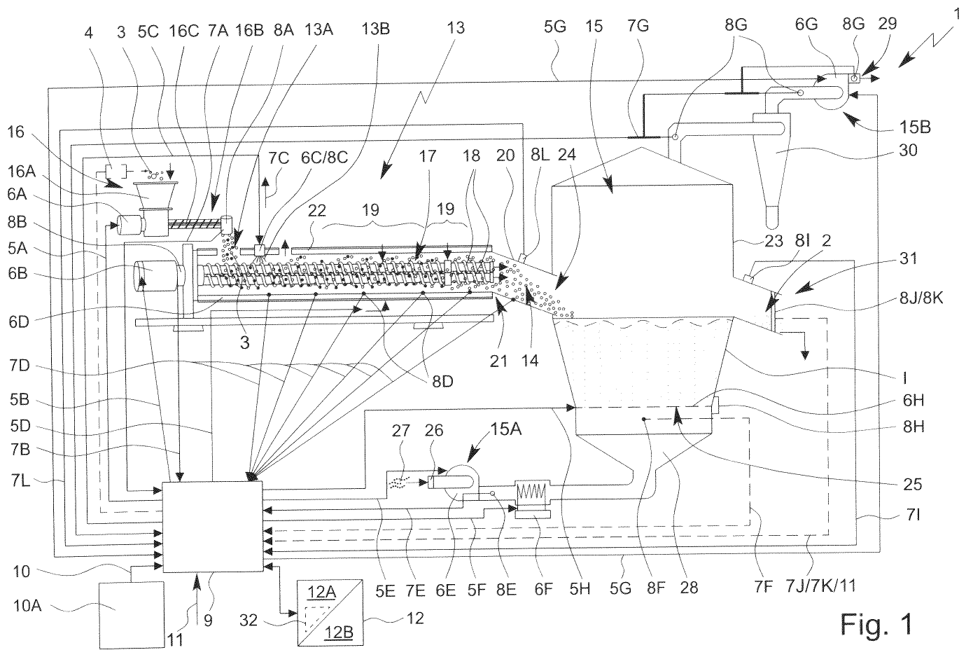

FIG. 1 shows a schematic section of a proposed installation 1 for the production of a formulation 2 from a feedstock 3. The installation 1 is preferably configured for carrying out a method for the production of the formulation 2 from the feedstock 3.

For the feedstock 3, feedstock parameters 4 are specified/preset, which represent a state of the feedstock 3, in particular a moisture, composition and/or a grain property, such as a particle size distribution.

The installation 1 is preferably configured to determine control parameters 5 of the installation 1, which are or represent manipulated variables for controlling actuators 6 of the installation 1.

Furthermore, state parameters 7 of the installation 1 are preferably determined or can be determined with the installation 1, for example via sensors 8, in particular sensor values or variables derived therefrom as state parameters 7, wherein the state parameters 7 each represent a state of the installation 1 which influences the production.

With a control device 9 of the installation 1, the actuators 6 of the installation 1 can be controlled in order to control or influence the production process for forming the formulation 3 from the feedstock 2.

Target formulation parameters 10 can be specified/preset, which represent the desired properties of the formulation 2 produced or to be produced, in particular a physical property such as a grain property, in particular particle size distribution, and/or a moisture.

The target formulation parameters 10 can be stored and/or kept available in a database 10A. The database 10A can be read out by the control device 9 and/or the target formulation parameters 10 can be retrieved by the control device 9 from the database 10A and used for the control.

Actual formulation parameters 11 can be provided or measured, which represent actual properties of the formulation 3 produced or to be produced, in particular a physical property such as a grain property such as the particle size distribution and/or a moisture. Actual formulation parameters 11 preferably are or have information about attributes of the formulation 2, which are measured in a separate analysis process, in particular separate from the installation 1, preferably not in-line and/or not in real time/delayed.

The installation 1, in particular the control device 9, is preferably configured to determine the control parameters 5 based on a model 12.

The proposed model 12 can alternatively or additionally be used independently of the control device 9 and/or without direct influence or a preferably automatic control of the actuators 6, preferably for a forecast and/or output of forecasted actual formulation parameters 11 (to be expected under given boundary conditions) of the formulation 2. For this purpose, one or more properties of the formulation 2 can be forecasted with the model 12 from one or more state parameters 7 and control parameters 5 and, preferably, feedstock parameters 4.

In one aspect of the present invention, the installation 1 has a granulator 13 for processing the feedstock 2 to form an intermediate product 14 and a dryer 15 for the intermediate product 14.

The dryer 15 is coupled to the granulator 13 in such a way that the intermediate product 14 is conveyed automatically and/or without interruption from the granulator 13 into the dryer 15. The granulation and drying preferably form a common, continuous process.

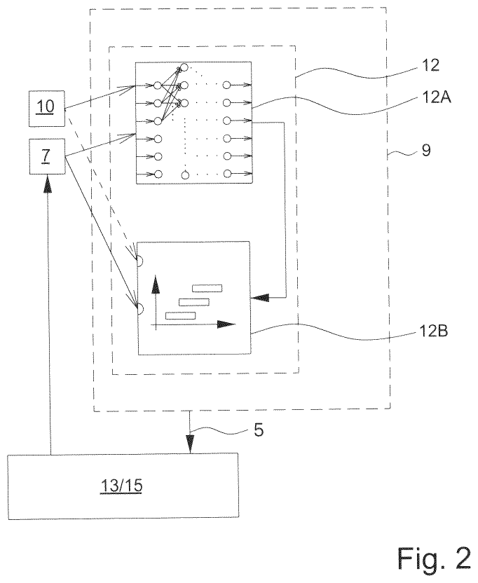

A simplified, block diagram-like view of control-relevant components of the installation 1 is shown in FIG. 2.

The control device 9 is preferably configured to control the installation 1 and/or the combination of granulator 13 and dryer 15. The control is preferably based on the model 12 which describes the behavior of the installation 1 and/or of the combination of granulator 13 and dryer 15 and, on the basis of at least one or more state parameters 7, makes it possible to determine control parameters 5 for controlling actuators 6 of the installation 1 and/or of the combination of granulator 13 and dryer 15. Here, it is particularly preferred that the model 12 takes into account the combination of granulation with the granulator 13 and the subsequent drying with the dryer 15.

With the model 12, the control parameters 5, in particular initially basic values of the control parameters 5, are preferably determined, preferably based on the state parameters 7 of the installation 1 and, further preferably, based on the feedstock parameters 4.

For this purpose, the model 12 can have a static part 12A, with which a base value is determined for the respective control parameter 5 taking into account feedstock parameters 4 and based on the state parameters 7, and the model 12 can have a dynamic part 12B, with which the (respective) base value can be adjusted, preferably optimized by means of a forecast.

In other words, the static part 12A of the model 12, which is preferably formed by means of machine learning, is used to determine base values (basic settings) of the control parameters 5, which can then be adjusted and finalized by means of the dynamic part 12B of the model 12, in order ultimately to be used for controlling the actuators 6.

The dynamic part 12B of the model 12 preferably effects a fine tuning of the base values and/or basic settings determined with the static part 12A of the model 12. Here, the base values of the control parameters 5 determined with the static part 12A are preferably independent of a transient behavior, i.e., the behavior at runtime, for example, under influence of environmental influences, tolerance changes, wear of the installation 1 and/or of the production process of the formulation 2.

In contrast thereto, the adjustment values determined with the dynamic part 12B for the control parameters 5 and/or correspondingly adjusted settings and/or control parameters 5 are those which take into account transient and/or runtime effects, for example via one or more forecasts. This is preferably done taking into account past developments, in particular by means of time-series forecasting.

For this purpose, adjustment values for the base values of the control parameters 5 and/or correspondingly adjusted settings and/or control parameters 5 can be determined, which preferably take into account a comparison of forecasted actual formulation parameters 11 with the target formulation parameters 10 and, by dynamic adjustment of the base values of the control parameters 5, approximate the actual formulation parameters 11 to the target formulation parameters 10, in particular on the basis of current state parameters 7 at runtime.

An advantageous peculiarity of this procedure is that the base values and adjustments can be determined by different methods. Alternatively or additionally, it is provided that at least the base values are determined by means of an AI and/or a machine learning method, preferably differently than the adjustments. Furthermore, it is particularly preferred that the determination of the base values also depends on the state parameters 7, i.e. base values as well as adjustments (correction terms) for determining adjusted and/or corrected base values are variable.

It is thus possible and preferred that the base values do depend on the state parameters 7, but are independent of the historical and forecasted development of the state parameters 7. By contrast, base values of the control parameters 5 determined with the static part 12A of the model 12 can be variable depending on the (current) state parameters 7, as well as correction terms and/or adjustments, which are determined with the dynamic part 12B of the model 12 and adjust and/or correct the base values.

In this case, the static part 12A of the model 12 differs from the dynamic part 12B of the model 12 preferably in that, with the static part 12A, the base values are determined or determinable without forecasts of the influence of state parameters 7 changing with changes in control parameters 5 and/or past changes in the state parameters 7, while the dynamic part 12B takes into account an influence of state parameters 7 changing with changes in control parameters 5 in the future and/or forecasts resulting from past changes in control parameters 5 or state parameters 7, further preferably taking into account previous changes.

In summary, the static part 12A of the model 12 differs from the dynamic part 12B of the model 12 preferably in that they implement different methods in order to determine, with or from the state parameters 7 and, preferably, taking into account the feedstock parameters 4, base values for the control parameters 5 in the case of the static part 12A and correction terms for the base values of the control parameters 5 or corrected/adjusted base values as final control parameters 5.

The state parameters 7 taken into account by the model 12 as input variables are preferably feedstock-influenced state parameters 7. Here, the model 12 preferably uses at least one feedstock-influenced state parameter 7 each of the granulator 13 and the dryer 15.

Optionally, the model 12 additionally takes into account, as input variable, an (exclusively) in-line measurable formulation property parameter 7K, which describes a physical or chemical property of the formulation 2 measurable in the uninterrupted, continuously running process.

Alternatively or additionally, the model 12 additionally takes into account, as input variable, an (exclusively) in-line measurable intermediate product property parameter 7L, which describes a physical or chemical property of the intermediate product 14 measurable in the uninterrupted, continuously running process. The measurement can be done with an intermediate product property sensor 8L, in particular an (NIR) sensor for determining an indicator for the moisture content (water content) of the intermediate product 14.

By contrast, taking into account further other parameters is not excluded. In addition, the definition of the model 12 can take into account parameters that are not measurable in-line, while the model 12 preferably does not require any parameters that are not measurable in-line as input variables in ongoing operation.

The static part 12A of the model 12 can implement an AI method and/or machine learning method, while the dynamic part 12B of the model 12 preferably implements another, preferably non-AI method and/or non-machine learning based method and/or no neural network 32. On the other hand, it is not excluded that both different methods are AI-based and/or machine learning-based or both are not.

It has surprisingly been found that the closed-loop control of the installation 1 is particularly reliable and accurate, thus the actual formulation parameters 11 are particularly close to the target formulation parameters 10, if the previously described aspects are combined with one another. In summary, it is thus surprisingly very particularly advantageous:
to control the combination of granulator 13 and dryer 15 or to make the control possible, and/or
in the case of direct coupling of the installation components without external and/or non-in-line analysis of the intermediate product 14, and/or
with the exclusion of any use of analysis results of the intermediate product 14 that are not measured or measurable in-line—thus not in the continuously running process and/or on the continuously moving intermediate product 14, and/or
in that base values of the control parameters 5 are determined/determinable with the static part 12A of the model 12 with a first, preferably machine-learning-based method, and/or
in that the base values of the control parameters 5 are determined/determinable without taking into account forecasts of the influence of state parameters 7 changing with changes in control parameters 5 in the determination, and/or
in that dynamic portions of the control parameters 5 are determined or determinable with the dynamic part 12B of the model 12; or when the base values are corrected with the dynamic portions or the installation 1 is configured therefor, and/or
in that the dynamic portions of the control parameters 5 are determined or determinable with the dynamic part 12B of the model 12 by means of another, preferably time-series-forecasting and/or non-machine learning-based method, than the method used or supported by the static part 12A of the model 12, and/or
in that the dynamic part 12B of the model makes and takes into account forecasts of the influence of state parameters 7 changing with changes in control parameters 5 or the installation 1 is configured therefor, and/or
in that the base values for the control parameters 5 are corrected and/or adjusted with the dynamic portions or correction terms are determined/determinable for this purpose or the installation 1 is configured therefor, and/or
in that the ultimate control parameters 5 are the base values adjusted with the dynamic portions and the installation 1 is controlled with these control parameters 5.

As shown in FIG. 1, the installation 1 can have a plurality of different actuators 6 in order to act directly or indirectly on the feedstock 3, whereby the intermediate product 14 and ultimately the formulation 2 are formed.

A control parameter 5 corresponds to the respective actuator 6, which can be or correspond to a manipulated variable for the actuator 6 in order to control the behavior of the actuator 6.

The actuators 6 of the granulator 13, which are controllable via corresponding control parameters 5, include one or more of the following actuators 6:
a feed conveyor drive 6A controllable by means of a feed conveyor drive control parameter 5A, preferably for metering and/or conveying the feedstock 3 to the granulator 13, and/or
a granulator drive 6B controllable by means of a granulator drive control parameter 5B, preferably for driving the granulating unit 17 and/or one or more screws, and/or
an injection device 6C controllable by means of an injection device control parameter 5C, preferably for injecting granulating liquid 13B, and/or
a granulator temperature control device 6D controllable by means of a granulator temperature control device control parameter 5D, preferably for controlling the temperature (heating and/or cooling) of parts of the granulator 13 coming into contact with the feedstock 3.

The actuators 6 of the dryer 15, which are controllable via corresponding control parameters 5, include one or more of the following actuators 6:
a supply air conveyor drive 6E controllable by means of a supply air conveyor drive control parameter 5E, preferably a fan, for supplying air 27 to the dryer 15, and/or
a supply air temperature control device 6F controllable by means of a supply air temperature control device control parameter 5F, preferably for controlling the temperature of the air 27, and/or
an exhaust air conveyor drive 6G controllable by means of an exhaust air conveyor drive control parameter 5G, preferably for extracting air 27 after the drying process, and/or
a fluidized bed drive 6H controllable by means of a fluidized bed drive control parameter 5H, preferably for rotating a carousel of the dryer 15.

The sensors 8 for measuring respectively corresponding state parameters 7 of the granulator 13 include one or more of the following sensors 8:
a feed conveyor sensor 8A for measuring a feed conveyor parameter 7A which characterizes the feed, in particular a throughput and/or a speed of the feed—preferably a conveying rate or metering representable in [kg/h]; and/or
a granulator sensor 8B for measuring a granulator parameter 7B which characterizes a functional property of the granulator 13, in particular a torque and/or a speed of its drive 6B, and/or a throughput—preferably a conveying rate of the granulator representable in [kg/h] or rotational speed [rpm] of the extruder/screws corresponding thereto; and/or
an injection sensor 8C for measuring an injection device parameter 7C which characterizes an addition of liquid 13B, in particular water and/or alcohol, to the feedstock 3 in the region of the granulator 13, in particular a throughput and/or a quantitative ratio in comparison to the feedstock—preferably a spraying rate representable in [g/min]; and/or
one or more granulator temperature sensors 8D for measuring one or more granulator temperatures 7D, in particular temperatures of the granulator 13 or (indirectly) of the feedstock 3 at different positions along a transport path for the feedstock 3 through the granulator 13—preferably in [° C.] or corresponding.

The sensors 8 for measuring respectively corresponding state parameters 7 of the dryer 15 include one or more of the following sensors 8:
a supply air conveyor sensor 8E for measuring a supply air conveyor parameter 7E which characterizes a supply air conveyance of the dryer 15, in particular a (mass) throughput, a pressure difference (corresponding thereto) and/or a speed of the supplied air 27—preferably representable in [m\hat{ }3/h]; and/or
a supply air sensor 8F for measuring a supply air parameter 7F which characterizes a property of the supplied air 27, in particular a temperature—preferably representable in [° C.]—and/or a moisture of the supplied air 27, preferably representable in %, wt %, or [° C.] for the dew point; and/or
an exhaust air sensor 8G for measuring an exhaust air parameter 7G which characterizes an exhaust air conveyance of the dryer 15, in particular a (mass) throughput, a pressure difference (corresponding thereto), a temperature of the air 27 discharged from the dryer 15 and/or a moisture thereof; and/or
a fluidized bed sensor 8H for measuring a fluidized bed parameter 7H which preferably characterizes a drying-relevant variable property relating to the fluidized bed of the dryer 15, in particular a speed such as a carousel speed of a carousel of the dryer—preferably representable in [rpm].

Alternatively or additionally to the use of sensors 8, in principle also properties of actuators 6 or control parameters 5 can partly be used to determine or derive one or more of the aforementioned or corresponding quantities. These can then be used as the basis for a control accordingly.

In the determination of feedstock-influenced state parameters 7, by contrast, a measurement by means of sensors 8 is mandatory. These state parameters 7 are also used as unit variables for the model 12.

Optionally as part of the installation 1, alternatively or additionally provided externally are a formulation temperature sensor 8I for measuring the formulation temperature 7I which characterizes a temperature of the formulation 2; and/or
a formulation outlet quantity sensor 8J for measuring a formulation outlet quantity parameter 7J which characterizes a property of the production and/or of the installation 1 with regard to the production of the formulation 2, in particular an outlet quantity of the formulation 2; and/or
a formulation property sensor 8K which characterizes an in-line measurable formulation property parameter 7K of the formulation 2, in particular a particle size, particle size distribution, granularity, and/or moisture, particularly preferably a particle size or particle size distribution (XD10, XD50, XD90) and/or a residual moisture and/or a drying loss of the intermediate product 14 during processing to the formulation 2.

The aforementioned control parameters 5, actuators 6, state parameters 7 and sensors 8 are particularly preferred examples with regard to the exemplary embodiment of a particularly preferred combination of a granulator 13—preferably (twin-) screw granulator 13—with a dryer 15—particularly preferably fluidized-bed dryer 15.

It is understood that other control parameters 5, actuators 6, state parameters 7 and sensors 8 are alternatively or additionally possible in other granulators 13 and/or dryers 15. The invention is thus preferably not restricted to the aforementioned control parameters 5, actuators 6, state parameters 7 and sensors 8. Furthermore, it is not necessary for all control parameters 5, actuators 6, state parameters 7 and sensors 8 to be implemented or used in the following. Different selections are possible here.

The production of the formulation 2 with the installation 1 is explained in more detail in the following with reference to the exemplary embodiment shown in FIG. 1. It is understood that the invention can in principle be implemented particularly preferably and advantageously with the installation 1 described in the following, but is not restricted thereto. In particular, it is not necessary for all described components of the installation 1 or steps carried out therewith also to be implemented.

For instance, it is conceivable to use the invention also in those cases in which other granulator technologies and/or dryer technologies are used. Therefore, a different type of granulator can be used than a twin-screw granulator 13. Alternatively or additionally, a different type of dryer can be used than a fluidized-bed dryer 15. Regardless of this, the invention has proven to be particularly advantageous in this context.

In the exemplary embodiment according to FIG. 1, the installation 1 has the granulator 13 for processing the feedstock 3 to form the intermediate product 14 as well as the dryer 15 for forming the formulation 2 from the intermediate product 14.

The granulator 13 has a feed conveyor 16 for feeding and/or metering the feedstock 3. The feed conveyor 16 can have in particular a funnel-shaped storage container 16A which holds the feedstock 3. Furthermore, the feed conveyor 16 can have a supply device 16B which supplies the feedstock 3 at a specific supply rate (quantity per time) to a granulating unit 17 of the granulator 13 coupled to the feed conveyor 16.

The feed conveyor 16 can have a drive 6A for the supply device 16B, in particular a motor for driving a worm or screw 16C. Generally, however, other principles of the feeding than with a screw 16C are also possible, such as a conveyor belt. The drive 6A is, as already explained above, preferably controllable by means of the feed conveyor parameter 7A. With the feed conveyor drive control parameter 5A, the supply rate can preferably be set.

The feed conveyor 16 preferably has one or more sensors 8A for measuring the throughput and/or a speed—preferably corresponding thereto.

The feed conveyor 16 is preferably followed by a granulating unit 17 of the granulator 13. The granulating unit 17 is configured to change a graining and/or particle size distribution of the feedstock 3. For this purpose, the granulating unit 17 can act on the feedstock 3 physically, in particular by kneading and/or milling/tumbling.

In the illustrative embodiment, the granulating unit 17 has at least one screw 18, preferably a twin screw. The screws 18 of the twin screw preferably engage in one another and transport the feedstock 3 while they act on it physically in order to change the graining and/or particle size distribution of the feedstock 3.

The granulating unit 17 and/or the screw(s) 18 can have different processing zones 19. In particular, different screw flight pitches and/or surfaces can be provided in order to achieve the desired processing of the feedstock 3.

The granulating unit 17 can have the granulator drive 6B in order to drive the granulating unit 17, in particular the screw(s) 18. As already explained above, the granulator drive 6B is controllable via the granulator drive control parameter 5B, preferably with regard to throughput and/or speed, in particular rotational speed of the screw(s) 18.

The granulator 13 preferably has the granulator sensor 8B with which a parameter representing the granulating speed and/or processing intensity can be measured, in particular a speed, a throughput or—very particularly preferably—a torque (of the granulating unit 17/of the screw(s) 18).

The granulator 13 can have an injection device 6C for injecting liquid 13B for the purpose of mixing with and/or admixture to the feedstock 3. This may be a sprayer, but alternatively also a dripper or generally a device for adding a liquid substance.

The injection device 6C is preferably arranged in the region of an inlet 13A and/or in the first half and/or in the first third of the transport path formed by the granulating unit 17 for the feedstock 3 between the inlet 13A and an intermediate product outlet 21 of the granulator 13 for the feedstock 3 processed with the granulator 13.

The granulator 13 preferably has at least one, but preferably more granulator temperature sensor(s) 8D for measuring the temperature 7D of the feedstock 3 or a corresponding temperature 7D of the granulator 15 or the housing 22 thereof at a wide variety of positions.

In the illustrative embodiment, more than three and/or less than ten granulator temperature sensor(s) 8D are provided for measuring the temperature 7D of the feedstock 3 or a corresponding temperature 7D of the granulator 15. The temperature sensor(s) 8D are preferably provided (at least substantially equidistantly) distributed along the transport path for the feedstock 3 in the granulator 15 and/or granulating unit 17.

A coupling device 20 preferably makes possible a continuous and/or interruption-free transfer of the intermediate product 14 from the intermediate product outlet 21, which can be formed by the housing 22 of the granulator 13, to an intermediate product inlet 24 of the dryer 15, preferably formed by a housing 23 of the dryer 15.

Preferably, it is not provided that the intermediate product 14 is stopped for the purpose of intermediate storage, at least not for more than one, two or five minutes. It is therefore in particular not provided to perform a sampling and analysis of the intermediate product 14 separate from the installation and/or delaying the production process or to wait. Rather, it is preferred in the sense of the present invention to supply the intermediate product 14 at least substantially without interruption and/or continuously to the dryer 14.

One or one of the granulator temperature sensor(s) 8D can be provided to measure a temperature 7D of the intermediate product 14 or a temperature 7D corresponding thereto, in particular of the granulator 15 in the region of the coupling device 20.

The dryer 15 preferably has a fluidized bed 25. Furthermore, the dryer 15 preferably has an air inlet 26 for the inlet of (process) air 27, an optional diffuser 28 for uniform charging of the fluidized bed 25 with the air 27 and an air outlet 29 for discharging the air 27 after passing through the fluidized bed 25. In the region of or in front of the air outlet 29, the dryer 15 optionally has a separator 30 such as a cyclone separator or filter for capturing particles of the intermediate product 14 and/or of the formulation 2 from the air 27.

Finally, the dryer 15 preferably has a formulation outlet 31 for discharging the formulation 2, i.e. the intermediate product 14 dried with the dryer 15. The dryer 15 dries the intermediate product 14 entering through the intermediate product inlet 24 with the air 27 and subsequently discharges the formulation 2 formed thereby through the formulation outlet 31, i.e. preferably after passing through the fluidized bed 25.

In order to supply the air 27 to the dryer 15, i.e. in particular to the fluidized bed 25, the dryer 15 preferably has a supply air conveyor 15A with a supply air conveyor drive 6E. This may be a fan or generally a device for transferring and/or for compressing air 27.

The dryer 15 can have the supply air conveyor sensor 8E with which a throughput of the air 27 or a quantity corresponding thereto can be measured. In particular, the supply air conveyor sensor 8E is a pressure sensor for measuring the air pressure on the discharge side and/or the side of the supply air conveyor drive 6E facing the fluidized bed 25 and/or a differential pressure sensor for determining a differential pressure across the supply air conveyor 15A. Alternatively or additionally, the supply air conveyor sensor 8E can be or have a quantity assigned to the supply air conveyor drive 6E, such as a rotational speed (fan rotational speed), a current consumption, a torque or the like.

The supply air conveyor 15A and/or its supply air conveyor drive 6E can be controlled by means of the supply air conveyor drive control parameter 5E, in particular with regard to a throughput, a rotational speed (of the fan), a current consumption and/or a pressure or differential pressure. The differential pressure can be a pressure across the supply air conveyor 15A, but alternatively or additionally also across the fluidized bed 25 or the like.

The air 27 is preferably temperature-controlled, in particular heated, before it is supplied to the fluidized bed 25 or another drying device of the dryer 15. For this purpose, the dryer 15 preferably has the supply air temperature control device 6F, preferably a heating register. The supply air temperature control device 6F can be controlled by means of the supply air temperature control device control parameter 5F or can be configured therefor.

The temperature and/or (relative) moisture of the conditioned air 27 brought into contact or to be brought into contact with the intermediate product 14 for the purpose of drying is preferably measured. For this purpose, the dryer 15 can have the supply air sensor 8F which measures the temperature and alternatively or additionally a (relative) moisture of the air 27 as supply air parameter 7F or is configured therefor.

The supply air sensor 8F can be provided between the fluidized bed 25 and the supply air temperature control device 6F or the supply air conveyor 15A and/or can measure the properties of the air 27.

After the air 27 has been brought into contact with the intermediate product 14 for the purpose of drying, the air 27 is discharged by the dryer 15. This is preferably done via the air outlet 29, which differs from a formulation outlet 31 for discharging the formulation 3.

The air 27 can be conveyed out of the air outlet 29 by means of the exhaust air conveyor 15B, in particular a (second) fan. For this purpose, the exhaust air conveyor drive 6G can be provided which effects the conveying and/or drives the exhaust air conveyor 15B.

The exhaust air conveyor 15B can be open-loop controlled or closed-loop controlled by means of the exhaust air conveyor drive control parameter 5G. In particular, it is provided that the exhaust air conveyor 15B is closed-loop controlled such that no air 27 escapes through the intermediate product outlet 21 and the formulation inlet 31. For this purpose, the conveyed quantity of the exhaust air conveyor 15B can correspond to or exceed the conveyed quantity of the supply air conveyor 15A.

By means of an exhaust air conveyor sensor 8G, a throughput, a speed, a temperature and/or moisture of the air 27 discharged from the dryer 15 after the drying process and/or a corresponding quantity such as a speed of the exhaust air conveyor drive 6G and/or blade wheel of the fan can be measured as an exhaust air parameter 7G.

The fluidized bed 25 can have or form a processing zone, in particular on the side facing away from the air inlet 26. The fluidized bed 25 and/or a structure delimiting it in the direction of the air inlet 26, such as a screen or perforated plate and/or carousel, can be drivable, in particular can be set in motion. For this purpose, the fluidized bed drive 6H can be provided which is controllable with the fluidized bed drive control parameter 5H.

With the fluidized bed sensor 8H, the fluidized bed parameter 7H can be measured which can characterize a property of the fluidized bed 25 such as a movement of the carousel.

After drying of the intermediate product 14 with the dryer 15, the processed feedstock 3 is output as formulation 2.

From the formulation 2, in this instance or thereafter, one or more actual formulation parameters 11 can be determined, i.e. parameters which describe physical or chemical properties of the produced formulation 2. This can be done in particular in-line, i.e., in the uninterrupted, running process. Alternatively, or additionally, however, actual formulation parameters 11 can also be determined subsequently by means of laboratory investigation. Actual formulation parameters 11 that are not measurable in-line are preferably taken as a basis for the model 12, so that the modeling takes into account actual formulation parameters 11 that are not measurable in-line. By contrast, actual formulation parameters 11 that are not measurable in-line are not used directly for the control of the installation 1 or as input variable for the control 9 and/or the model 12.

The installation 1 can have one or more sensors 8 for in-line characterization of properties of the formulation 2. These include one or more of the formulation temperature sensor 8I for measuring a formulation temperature 7I, the formulation outlet quantity sensor 8J for measuring a formulation parameter 7J which describes the outlet quantity and/or the throughput (mass flow) of formulation, and/or the formulation property sensor 8K which measures one or more properties of the formulation 2 and outputs as formulation property parameter 7K, preferably the (relative) moisture and/or residual moisture and/or the drying loss.

The formulation parameters 10, 11 are preferably at least one parameter characterizing particles of the formulation 2, such as a particle size or particle size distribution (XD10, XD50 and/or XD90) or a quantity corresponding thereto.

Alternatively or additionally, the formulation parameters 10, 11 are preferably a (relative) moisture and/or residual moisture and/or a drying loss (LoD—loss on drying) and/or a quantity corresponding thereto.

The additional formulation parameters 10, 11 can be determined as required by the formulation temperature sensor 8I, the formulation outlet quantity sensor 8J and/or the formulation property sensor 8K and/or as formulation temperature 7I, formulation outlet quantity parameter 7J and/or formulation property parameter 7K.

In principle, corresponding measured variables, such as the particle size or particle size distribution (XD10, XD50 and/or XD90) of the formulation 2, can be used to form the model 12. However, it is not mandatory or provided in any case to determine corresponding quantities in ongoing operation of the installation 1 or to supply said quantities to the control thereof.

Optionally, but preferably, an in-line measurable property, in particular moisture, of the intermediate product 14 can be determined from the intermediate product 14 by means of an intermediate product (moisture) sensor 8L as intermediate product property parameter 8L. If provided, this can also be taken into account in the model 12, in particular used as input variable, and/or (additionally) used as basis for the control of the installation 1.

Actual formulation parameters 11 can be measured downstream of the installation 1. This will be discussed in more detail in the following. However, it should already be mentioned at this point that a parameter characterizing the shape, size or distribution of particles of the formulation 2 is preferably also measured in-line.

By contrast, a particle-characterizing measurement of the intermediate product 14 is preferably avoided.

In connection with the exemplary embodiment according to FIG. 1, various sensors 8 for determining state parameters 7 of the installation 1 have been described. However, it is not mandatory that all sensors 8 are provided and/or state parameters 7 are used.

Preferably, at least two or at least three state parameters 7 and/or sensors 8 are used respectively for the granulator 13 and the dryer 15.

The control parameters 5 for the installation control, which particularly preferably can be dynamically set for the installation control, the selection of which can represent an independent concept of the invention for the further aspects of the invention, include:

On the part of the granulator 13:
the feed conveyor drive control parameter 5A, preferably characterizing a metering of the feedstock 3; and/or
the granulator drive control parameter 5B, preferably characterizing an extruder speed of an extruder of the granulator 13 which can form a granulating unit 17 of the granulator 13, or a (other) quantity corresponding to a conveying and/or processing speed of the granulator 13; and/or
the injection device control parameter 5C, in particular a spraying rate characterizing the quantity per time of supplied liquid 13B.

On the part of the dryer 15:
the fluidized bed drive control parameter 5H, preferably characterizing a speed of a carousel of the dryer 15;
the supply air conveyor drive control parameter 5E, preferably representing an inlet-side supply air flow, and/or
the supply air temperature control device control parameter 5F, in particular representing the temperature of the inlet-side supply flow of the air 27.

The particularly preferred parameters 3, 7 as input variables for the installation control and/or the model, the selection of which can represent an independent aspect of the invention for the further aspects of the invention, include:

On the part of the granulator 13:
the granulator parameter 7B, in particular characterizing an extruder torque of the extruder of the granulator 13 or another parameter of the granulator drive 6B dependent on the consistency and/or conveying rate of the feedstock 3 being processed; and/or the one or more granulator temperature(s) 7D, in particular characterizing one or more temperatures of the feedstock 3 being processed in the granulator 13 at preferably different positions along a material flow of the feedstock 3 in the granulator 13 or temperatures corresponding thereto; and/or
the granulator temperature 7D and/or the intermediate product property parameter 7L, which is or corresponds to the temperature of the intermediate product.

On the part of the dryer 15:
the exhaust air parameter(s) 7G, in particular characterizing the exhaust air temperature and/or (relative) moisture and/or throughput (for example represented via a pressure difference) of the exhaust air 27; and/or
the formulation outlet quantity parameter 7J, in particular characterizing the outlet quantity and/or a pressure difference in connection with the output of the formulation, for example via a filter or screen.

On the part of the feedstock 3:
the feedstock parameter 4, preferably characterizing a moisture and/or granularity of the feedstock 3.

Optionally can be additionally taken into account for the control of the installation 1, on the part of the formulation 2:
the formulation property parameter 7K, preferably characterizing a particle size distribution, in particular D10, D50 and/or D90, and/or a drying loss.

The use of the aforementioned parameters 4, 5, 7 can be supplemented by one or more of the parameters 4, 5, 7 discussed above and below.

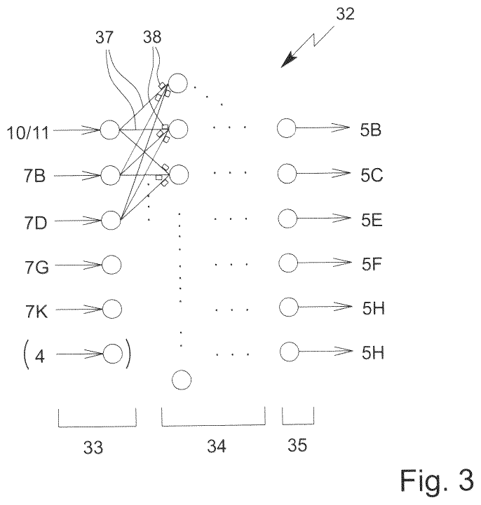

FIG. 3 shows a simplified, schematic view of an artificial neural network 32 for the determination of control parameters 5 for controlling the installation 1 and/or the actuators 6 thereof.

According to an aspect of the present invention, the model 12, in particular the static part 12A of the model 12, is formed by the artificial neural network 32 or comprises the artificial neural network 32. An example of the structure of the artificial neural network 32 is reproduced in FIG. 3.

The artificial neural network 32 comprises, in an input layer 33, nodes 36 in the form of input nodes which correspond to one or more of the feedstock parameters 4, to one or more of the state parameters 7 of the granulator 13, to one or more of the state parameters 7 of the dryer 15 and/or to one or more of the target formulation parameters 10.

The artificial neural network 32 preferably comprises nodes 36 in one or more hidden layers 34, via which the input layer 23 can be linked to an output layer 35.

The artificial neural network 32 can comprise nodes 36 in the form of output nodes in an output layer 35 which correspond to one or more of the control parameters 5.

The nodes 36 of different layers 33, 34, 35 can be connected to one another by means of edges 37. The nodes 36 can form a graph by means of the edges 37. The nodes 36 and/or edges 37 preferably have weights 38 which specify the links of the nodes 36 representable by means of the edges 37.

The artificial neural network 32 is preferably trained with data sets which consist of different combinations of the feedstock parameters 4, state parameters 7 and control parameters 5 as well as actual formulation parameters 11 occurring when these parameters are given.

The training data sets each represent a stationary state of the installation 1, in which the actual formulation parameters 11 and state parameters 7 have assumed an at least substantially static value based on constant control parameters 5 and feedstock parameters 4.

The artificial neural network 32 is preferably trained by subjecting the input nodes 36 to at least one, preferably more, preferably feedstock-influenced, state parameters 7 of the granulator 13 and at least one, preferably more, corresponding, preferably feedstock-influenced, state parameters 7 of the dryer 15 of the respective training data set.

The input nodes (nodes 36 in the input layer 33) are preferably furthermore each subjected to one or more corresponding actual formulation parameters 11 and to one or more corresponding feedstock parameters 4 of the respective training data set.

By specifying the parameters 4, 5, 7, 11 of a training data set in the input layer 33, control parameters 5 (values of the neural network 32) result at the output nodes (nodes 36 of the output layer 35), wherein preferably from the control parameters 5 errors are determined by comparison with the control parameters 5 of the respective training data set and the errors are reduced and/or compensated by adjustment of the weights 38 of the artificial neural network 32, preferably by means of back propagation and/or successively.

In order then ultimately to determine control parameters 5 by means of the neural network 32, instead of actual formulation parameters 11, target formulation parameters 10 together with further current parameters 4, 7 are specified/provided to the artificial neural network 32 (at the input layer 33), whereupon control parameters 5 result (at the output layer 35) for the control of the installation 1, on the basis of which the installation 1 is controllable or (automatically) controlled.

The parameters 4, 5, 7, 10 for which nodes 36 are provided are preferably at least:

Preferably in the input layer 33:
at least one node 36 for at least one corresponding feedstock parameter 4, preferably the moisture of the feedstock 3 and/or a property of the particles of the feedstock 3, in particular its particle size distribution; and/or
a node 36 for the granulator parameter 7B, in particular the screw torque and/or extruder torque of the granulator 13; and/or
a node 36 for the granulator temperature 7D, in particular a plurality of nodes 36 for a plurality of granulator temperatures 7D; and/or
a node 36 for an exhaust air parameter 7G, in particular for the temperature and moisture of the air 27 and/or the pressure difference of the air 27 over the separator 30;

Preferably in the output layer 35:
a node 36 for a granulator drive control parameter 5B; and/or
a node 36 for an injection device control parameter 5C; and/or
a node 36 for a fluidized bed drive control parameter 5H; and/or
a node 36 for a supply air conveyor drive control parameter 5E; and/or a node 36 for a supply air temperature control device control parameter 5F.

Optionally, nodes for one or more of the following state parameters 7 are provided in the input layer 34:
a node 36 for an injection device parameter 7C;
a node 36 for a supply air parameter 7F;
a node 36 for a supply air conveyor parameter 7E;
a node 36 for a fluidized bed parameter 7H;
a node 36 for a formulation temperature 7I;
a node 36 for a formulation outlet quantity parameter 7J; and/or
a node 36 for a formulation property parameter 7K,
and/or for one or more of the following control parameters 5 in the output layer 35 are provided:
a node 36 for a feed conveyor drive control parameter 5A;
a node 36 for a granulator temperature control device control parameter 5D; and/or
a node 36 for an exhaust air conveyor drive control parameter 5G.

Thus, it is preferred that the state parameters 7, to which a node 36 in each case corresponds, comprise the granulator parameter 7B, in particular a granulating unit torque of the granulator 13, one or more of the granulator temperatures 7D at different positions along a transport path of the granulator 13 for the feedstock 3, the formulation temperature 7I, in particular a temperature of the formulation 2 at the formulation outlet 31 of the dryer 15, a formulation property parameter 7K, in particular the moisture, in particular relative moisture, of the formulation 2 at the formulation outlet 31 of the dryer 15, and/or an exhaust air parameter 7G, in particular characterizing a pressure loss over a filter, here by way of example (cyclone) separator 30, of the dryer 15.

The artificial neural network 32 is preferably configured by training to generate, from the parameters 4, 7, 10 fed to the nodes 36 of the input layer, control parameters 5 with which the installation 1 and/or the combination of granulator 13 and dryer 15 is controllable. For this purpose, the control parameters 5 generated by the artificial neural network 32 are preferably, but not necessarily, optimized by means of the dynamic model 12B before they are used as the basis for the control of the installation 1.

With the model 12, the control parameters 5 are preferably determined only or primarily on the basis of the feedstock parameter(s) 4, state parameter(s) 7 and target formulation parameter(s) 10.

It remains that also in this case, properties of the intermediate product 14 that are not measurable in-line preferably remain out of consideration. Preferably, at most the temperature and/or moisture of the intermediate product 14 are taken into account.

The model 12 preferably takes into account future effects of changes in state parameters 7 on the actual formulation parameters 11, preferably by forecasts. For this purpose, the dynamic part of the model 12 is preferably configured to take into account changes in the actual formulation parameters 11 caused by long-term effects.

The control 9 preferably takes into account future effects of changes in the control parameters 5 on the actual formulation parameters 11 and/or state parameters 7, preferably by forecasts. In particular, the dynamic part of the model 12 is configured to pre-compensate for changes in the actual formulation parameters 11 caused by long-term effects.

For this purpose, the model 12 can use forecasts about future developments of the actual formulation parameters 11 as a basis for the determination or an adjustment of the control parameters 5. For this purpose, the model 12 can have, in addition to the static part 12A, with which a base value is determined for the respective control parameter 5 from the state parameters 7 and, preferably, the feedstock parameters 4 and the target formulation parameter(s) 10, the dynamic part 12B, with which the base value is optimized by means of a forecast.

With the dynamic part 12B of the model 12, it is thus possible, based on feedstock parameters 4 and current state parameters 7, to forecast a change in actual formulation parameters 11 in the event of a change in the current state parameters 7 and, based on this forecast, the base values for the control parameters 5 can be adjusted and the installation 1 can be controlled with the adjusted control parameters 5.

The model 12, in particular the dynamic part 12B of the model 12, is thus configured to forecast long-term effects of changes in the control parameters 5 on the actual formulation parameters 11 and/or state parameters 7. The control 9 is thus configured by the model 12 to control the installation 1 and/or combination of granulator 13 and dryer 15 while compensating for the long-term effects. Thus, surprisingly and despite the direct coupling of granulator 13 and dryer 15, a production can be achieved while maintaining small/admissible deviations of the actual formulation parameters 11 from the target formulation parameters 10.

If only a forecast of properties of the formulation 2 is desired or realized with the model 12, the model 12 and/or the artificial neural network 32 can be constructed differently than in the case of a preferably fully automatic control by means of the model 12, namely preferably in such a way that properties characterizing the formulation 2, in particular therefore one or more forecasted actual formulation parameters 11, can be determined and/or output.

In this case, with the model 12, one or more actual formulation parameters 11 are preferably determined and/or forecasted only or primarily on the basis of the feedstock parameter(s) 4, state parameter(s) 7 and specified/preset control parameters 5.

With the dynamic part 12B of the model 12, it is thus possible, preferably based on feedstock parameters 4, current state parameters 7 and/or control parameters 5 and/or their base values, to forecast a change in actual formulation parameters 11 taking into account a change in the state parameters 7 accompanying a change in control parameters 5 and, based on this forecast, the resulting actual formulation parameters 11 can be forecasted and, preferably, output.

The model 12, in particular the dynamic part 12B of the model 12, is thus preferably configured to forecast long-term effects of changes in the state parameters 7 on the actual formulation parameters 11. As a result, surprisingly and despite the direct coupling of granulator 13 and dryer 15, with the forecasted actual formulation parameters 11, the user can be given an indicator in order to select suitable control parameters 5.

Regardless of whether control parameters 5 or forecasted actual formulation parameters 11 are determined by the model 12, properties of the intermediate product 14 that are not measurable in-line preferably remain out of consideration, for example a physical property of the intermediate product 14, which characterizes particles of the intermediate product 14. In particular, all properties of the intermediate product 14 remain out of consideration, apart from the temperature and the moisture of the intermediate product 14.

The model 12 therefore preferably takes into account at most those parameters of the feedstock 2 being processed and/or the intermediate product 14 that can be measured in-line, thus requiring no sampling and analysis separate from the installation.

As already mentioned, the model 12 can alternatively be configured to forecast actual formulation parameters 11. In this case, the model 12 and/or the artificial neural network 32 is configured differently from the artificial neural network 32 for determining the control parameters 5.

In the case of determining forecasted actual formulation parameters 11 with the model 12, the parameters 4, 5, 7, 10 for which nodes 36 are provided are preferably at least:

Preferably in the input layer 33:
a node 36 for at least one corresponding feedstock parameter 4, preferably the moisture of the feedstock 3 and/or a property of the particles of the feedstock 3, in particular its particle size distribution; and/or
a node 36 for a granulator parameter 7B, in particular a screw torque and/or extruder torque of the granulator 13, and/or
a node 36 for the granulator temperature 7D, in particular a plurality of nodes 36 for a plurality of granulator temperatures 7D; and/or
a node 36 for an exhaust air parameter 7G, in particular for the temperature and moisture of the air 27 and/or the pressure difference of the air 27 over the separator 30; and/or
a node 36 for a granulator drive control parameter 5B; and/or
a node 36 for an injection device control parameter 5C; and/or
a node 36 for a fluidized bed drive control parameter 5H; and/or
a node 36 for a supply air conveyor drive control parameter 5E; and/or
a node 36 for a supply air temperature control device control parameter 5F.

Preferably in the output layer 35:
one or more nodes 36 for (respectively) a (forecasted) actual formulation parameter 11.

Optionally, nodes 36 for one or more of the following state parameters 7 are provided in the input layer 34:
a node 36 for an injection device parameter 7C and/or
a node 36 for a supply air parameter 7F and/or
a node 36 for a supply air conveyor parameter 7E and/or
a node 36 for a fluidized bed parameter 7H and/or
a node 36 for a formulation temperature 7I and/or
a node 36 for a formulation parameter 7J and/or
a node 36 for a formulation property parameter 7K and/or
a node 36 for a feed conveyor drive control parameter 5A and/or
a node 36 for a granulator temperature control device control parameter 5D and/or
a node 36 for an exhaust air conveyor drive control parameter 5G.

In this case, the artificial neural network 32 can be trained with corresponding and/or the same data sets as the artificial neural network 32 for determining the control parameters 5.

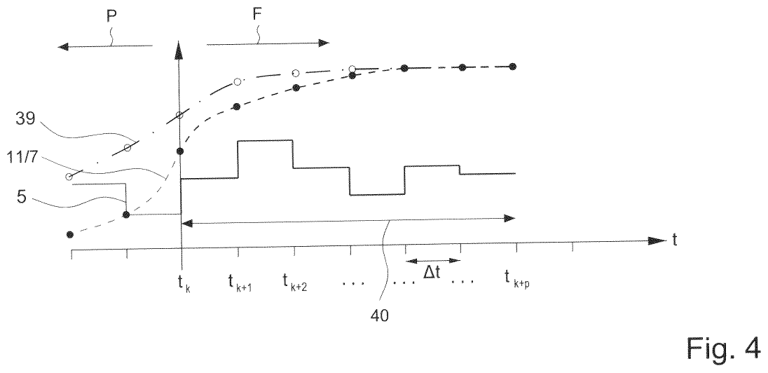

FIG. 4 shows a schematic diagram of past and forecasted courses of one or more parameters 5, 7, 10 and 11.

To the left of the Y-axis representing a value of a parameter 5, 7, 11, as indicated by an arrow for the past P, is the past development of the one or more parameters 5, 7, 11 as well as the past course of a reference trajectory 39 and a measured parameter 7, 11. Furthermore, as indicated by an arrow for the future F, the future development thereof is shown in a forecast horizon 40.

According to the proposal, it can be provided that one or more of the parameters 5, 7, 10 is changed at discrete times tₖ₊ₚ and/or a change is forecasted. In the illustrative embodiment, the times tₖ₊ₚ are spaced apart from one another by a sampling time/scan time Δt. In principle, however, it is not mandatory that the sampling time Δt is constant, even if this is possible, and the sampling time Δt can be selected to be small, so that the course of the parameter or parameters 5, 7, 11 can be at least substantially continuous.

As can be seen in FIG. 4, it is possible that, as a result of the forecast of the parameter or parameters 5, 7, 11, different and also both rising and falling courses can occur, with the aim of approximating a measurement value such as the state parameter 7 and/or the actual formulation parameter 11 to the reference trajectory 39.

Ultimately, the course according to FIG. 4 represents a possible system behavior, which takes into account an advantageously optimized control of the installation 1 and/or of the production process by means of the model 12 and the past development taken into account therewith as well as the development of different quantities forecasted for the future.

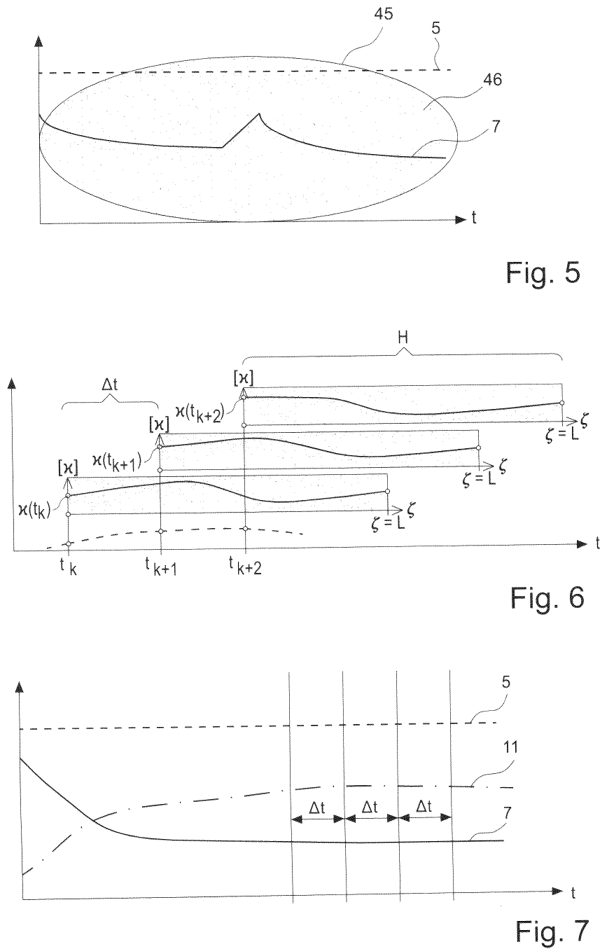

FIG. 5 shows a schematic diagram of the result of a control with constant control parameters 5 over time. In a preset, preferably fixed time window 41, one or more state parameters 7 result from the at least substantially constant control parameter(s) 5. Here, the state parameter(s) 7 preferably asymptotically approach a fixed value, taking into account quality dynamics 42, which can be measured at discrete times.

On the basis of the constant control parameter 5 or a combination of the constant control parameters 5, and the state parameter(s) 7 resulting therefrom, the static part of the model 12A can be determined. In particular, a machine-learning-based model 12A of the static installation behavior can be generated on this basis. This can, as explained above, be an artificial neural network 32, but in principle other machine-learning-based methods are also possible.

FIG. 6 shows a schematic diagram of forecasts offset in time with respect to one another over time t. At each time tₖ₊ₚ, future measurement values K depend on earlier developments and current control parameters 5 and/or state parameters 7. Such a regression can be solved by means of a time-series forecast, as indicated in FIG. 6. Shown are courses of the measurement values κ at different relative times ζ, which are offset in time with respect to one another by a time difference and/or sampling time Δt and by means of which it is indicated that these are forecasts of the time series.

Specifically, it is possible and preferred that forecasts made by means of the model 12 are renewed at regular intervals Δt. As soon as the state of the installation 1 has changed and thus one or more state parameters 7 deviate from previous values, it is thus possible—preferably by means of the model 12—to generate a changed and/or adjusted forecast, in particular of one or more control parameters 5, state parameters 7 and/or actual formulation parameters 11.

FIG. 7 shows a schematic diagram of process properties over time. The basic idea is to combine the static behavior, as explained for example with reference to FIG. 5, with the dynamic behavior and forecasted developments taking into account past courses, so that, as shown by way of example in FIG. 7, a process with static base values, preferably thus determined by the static part 12A of the model 12, is started and thereafter during the production by repeated, iterative optimization, in particular by means of a time-forecasting approach, the desired attributes, in particular thus actual formulation parameters 11, can be achieved in a short time and then at least substantially maintained.

In principle, it is thus possible to determine the control parameters 5 on the basis of the static part 12A of the model 12 at the start of the continuous processing of the feedstock 3 to form the formulation 2, and to easily readjust/update the control parameters 5 in ongoing operation of the installation 1 by means of the dynamic part 12B of the model 12. This is indicated in time in the time sections in FIG. 7 in the region in which the sampling times Δt are plotted.

It is thus possible that the installation 1 initially reaches a quasi-steady state before the readjustment is activated by means of the dynamic part 12B of the model 12 and then preferably sets in by means of a time-series forecasting at the interval of the respective sampling time Δt. In principle, however, other control strategies are also possible.

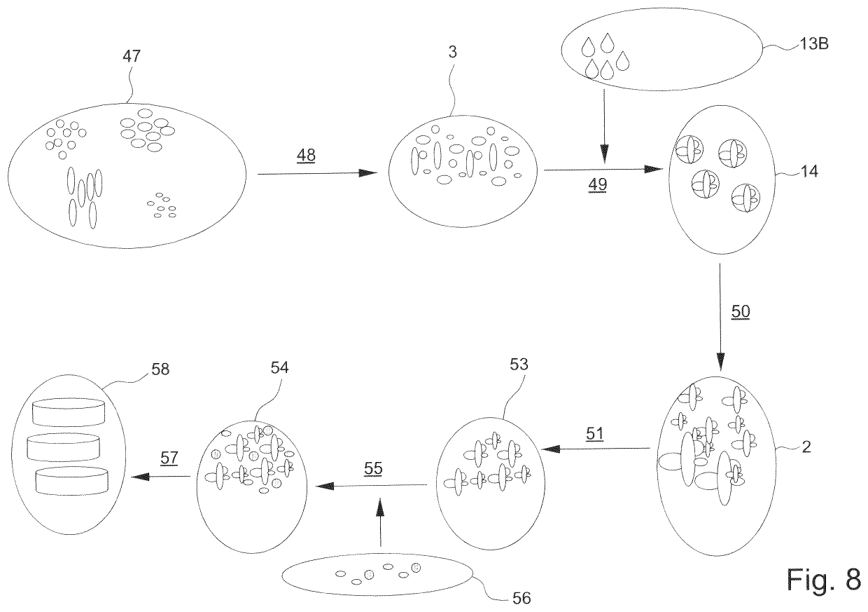

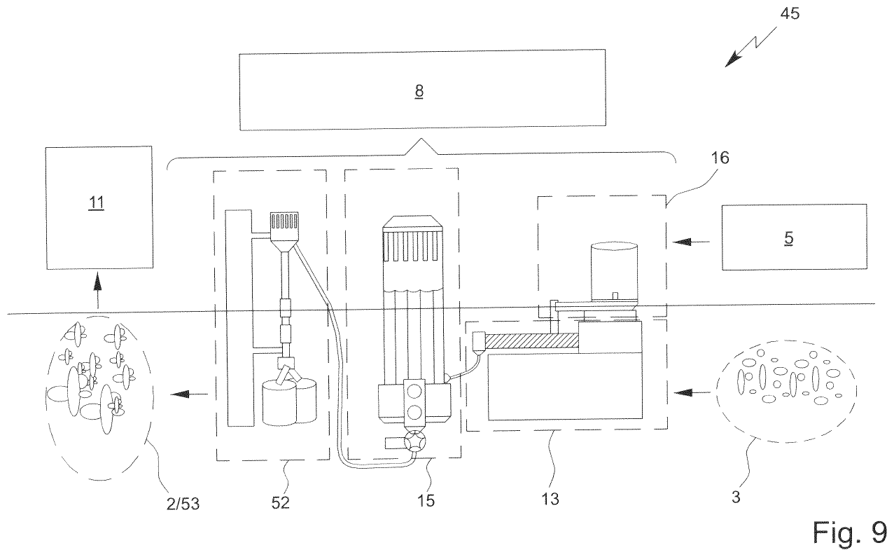

The proposed installation 1 can be used particularly advantageously in a system 45 for the production of tablets. An extended method is shown for this purpose in FIG. 8 with reference to a schematic flow diagram. In the system 45, components for processing the feedstock 3 and/or for post-processing the formulation 2 such as a preferably pneumatic conveyor installation 46 can partly be provided, which add additional functions to the installation 1, as will now be explained in more detail subsequently with reference to FIG. 9.

FIG. 9 shows an installation 1 embedded in a system 45. The preparation of the feedstock 3 can be upstream of the installation 1. In the illustrative embodiment according to FIG. 9 and with reference to the method according to FIG. 8, the feedstock 3 is produced from components 47 of the feedstock 3 by sieving and/or mixing in a preparation step 48, in the present case by way of example and also overall preferably a powder mixture of the components 47.

In a granulating step 49 preferably taking place continuously according to the proposal with the subsequent drying step 50, the feedstock 3 is subsequently processed by means of the granulator 13 to form the intermediate product 14, preferably with addition of liquid 13B, also called granulating liquid. In the continued continuous process, the intermediate product 14 is subsequently dried by means of the dryer 15, whereby ultimately the formulation 2 is produced. With regard to the continuous processing of the feedstock 3 to form the formulation 2, reference is additionally made to the previous sections.

Subsequently, the formulation 2 can be further processed. In one or more post-processing steps 51, the formulation 2 can be sieved, for example in a post-processing device 52, in particular for particle selection, to form a post-processed (in particular sieved) formulation 53. Alternatively or additionally, the formulation 2 and/or post-processed formulation 53 is mixed to form a final mixture 54 in a mixing process 55 with additives such as disintegrants and/or binders 56.

Ultimately, an administration form 58, in particular one or more tablets, can be produced from the formulation 2 and/or the post-processed formulation 53 or the final mixture 54 by means of a tableting process 57, in particular a compression.

The proposed, preferably pharmaceutical process concentrates on the continuous process steps of the production of solid oral administration forms, as explained by way of example with reference to a schematic flow diagram in FIG. 8 and in the following additionally with reference to the system 45 from FIG. 9.

After an optional, preferably batch-wise, preparation of a homogeneous powder premix of components 47 as feedstock 3, preferably by sieving and/or mixing of powders forming the feedstock 3 as components 47, the intermediate product 14, preferably wet granulate (wet or moist granulate), is formed in a continuous granulation step with the granulator 13, preferably a so-called twin-screw granulation (TSG). For this purpose, liquid 13B can be added to the feedstock 3 in the granulator 13 and/or during the granulation process. This can be done with the injection device 6C.

The intermediate product 14 is preferably transferred in a continuous product stream directly into the continuously operating dryer 15, here a fluidized-bed dryer. After the two continuous process steps of granulation and drying, the formulation 2, preferably a dry granulate (dried wet granulate), is obtained.

The formulation 2 is optionally and preferably subsequently sieved (to form the post-processed formulation 53) and/or mixed with an extragranular phase (an additive/disintegrant and/or binder 56) in order to obtain the final mixture 54. This final mixture 54 (end mixture) preferably forms the starting material for a tableting process 57, is used for tabletting, or the system 45 is designed for this purpose.

Thus, preferably, the two continuous process steps of granulation and drying are provided as installation 1. By contrast, the system preferably combines the installation 1 to form a total of at least three, in particular four, different process units and/or production steps.

The process units of charging and twin-screw wet granulation are responsible for the continuous granulation process. The two following process units—continuous fluidized-bed drying and pneumatic conveyor system—are responsible for the continuous drying process.

In total—preferably six—control parameters 5 (main input variables) are defined for the control/control device 9 of the continuous granulation and drying installation 1 and/or are used for the control.

The at least two, preferably at least three, control parameters 5 and/or main input variables for the granulator 13 are or comprise preferably the dosing quantity/metering quantity [kg/h] (of the feedstock 3), the extruder rotational speed [rpm] (of the granulator 13) and/or the spraying rate [g/min] (of the injection device 6C).

For the dryer 15, the at least two, preferably at least three, control parameters 5 and/or main input variables are or comprise the supply air flow [m³/h] (into the dryer 15), the supply air temperature [° C.] (of the supply air into the dryer 15) and/or the carousel rotational speed [rpm] (of the dryer 15 and/or of a carousel thereof).

Since in total preferably at least six main input parameters and/or control parameters 5 are defined for the installation 1 or are used for the control, it is easy to understand that the understanding of the relationships, the control and the monitoring of such a multifactorial installation 1 can represent a challenge.

A schematic view of the material flow and data flow of the continuous granulation and drying installation 1 and/or of the system 45 formed therewith is shown in FIGS. 8 and 9.

With regard to the material flow, it is preferred that the system 45 operates according to a bin-to-bin approach and/or is configured for this purpose. This means that a first container feeds a preferably homogeneous powder premix as feedstock 3 into the continuous line (from granulator 13 and dryer 15) in order to obtain the dried granulate as formulation 2 in a second container after the two continuously running process steps—preferably twin-screw wet granulation and fluidized-bed drying.

The powder premix as feedstock 3 is preferably prepared batch-wise by the system 45. The further processing of the dry granulate (formulation 2) is preferably also done batch-wise as shown by way of example in FIG. 9.

In contrast to a fully continuous production (from the raw material to the finished tablets), this bin-to-bin method offers a higher flexibility, since the system 45 itself can be used modularly. One advantage is thus to embed the continuous method of granulation and drying into a bin-to-bin method in order to be able to achieve the aforementioned advantages with a high flexibility at the same time.

Preferably, three types of data are taken into account for controlling the installation 1 and/or the system 45.

The first are control parameters 5 and preferably comprises the six relevant control parameters 5, as already described above.

The second type of data are state parameters 7 relating to process states (e.g. temperatures, pressure losses, torques) which can be measured via a plurality of sensors 8 in the entire installation 1, preferably in an online and/or real-time mode.

The third type of data preferably comprises one or more feedstock parameters 4 and/or actual formulation parameters 11. These critical material attributes (of the feedstock 3, of the intermediate product 14 and/or of the formulation 2) are preferably measured as in-process inspection, preferably separately from the installation 1, discontinuously and/or on the basis of random samples and therefore form the third type of data with a time delay.

Preferably, a complete data set with all three data types forms the basis for the creation of the model or models 12 which are used within the scope of the invention.

In particular, two relevant aspects can be emphasized as essential surprising advantages of the continuous granulation and drying installation 1 which are achieved by the invention, even if these are not mandatory:

The multifactorial interaction of—for example six—main input variables (control parameters 5 and state parameters 7 which can be influenced directly by control parameters 5 and/or are uninfluenced by the feedstock 3) and the resulting output variables. The resulting state parameters 7 and/or feedstock-influenced state parameters 7 (for example 10 states; measured online and in real time) and the material attributes (preferably attributes characterizing the feedstock 3 and the formulation 2 as described with, for example, a total of four material parameters and/or actual formulation parameters 11; measured offline and with a time delay) can be defined as output variables.

Furthermore, the continuous granulation and drying installation 1 preferably combines different (continuous) process steps. Therefore, the process parameters and/or control parameters 5 of a process unit also influence the process states and/or state parameters 7 of the following units and, at the end, also the material properties and/or actual formulation parameters 11.

If these two aspects are taken into account, it becomes clear that an optimum manual control of the process represents a challenge and hardly leads to a good result. For this reason, a great advantage of the preferred machine-learning-based method, which provides forecasts and control adjustments to this continuous process, becomes clearly visible.

In summary, the following aspects can be implemented individually or in different possible combinations in the installation 1:
integrated continuous process: not a completely continuous process (from the active substance and the auxiliaries up to the end product), but replacement of at least two traditional batch processes by one continuous process step; combination of batch processes with continuous steps=integrated continuous process
use of a flat-bottom metering device, which makes a very precise and easily controllable mass flow possible, as feed conveyor 16;
use of a twin-screw wet granulation installation as granulator 13;
use of a continuous fluidized-bed dryer as dryer 15 makes possible a dual functionality; the fluidized-bed dryer can be converted into a fluidized-bed granulator. This modular and more variable system makes possible a dual use of the main equipment, whereby the productivity of the installation can be increased, the footprint of the installation can be used efficiently and downtimes can be reduced;
use of a special, continuous dryer 15 with a slowly rotating carousel which divides the large fluidized-bed chamber into a plurality of small chambers, whereby the formation of partial batches is avoided;
reduction of the possible mass build-up of wet granulate (intermediate product 14) by shorter transport paths and by avoiding valves for wet granulate;
in-line data generation of NIR probes and/or particle size measurement system (intermediate product sensor 8L/formulation property sensor 8K) for measuring material properties of wet granulate (intermediate product 14) and/or dry granulate (formulation 2).

The invention relates furthermore to a computer program product or computer-readable storage medium, comprising commands which, when the program is executed by a computer, cause the latter to carry out the proposed method/the steps of the method or parts thereof.

Individual aspects of the present invention can be implemented separately from one another, but also in different combinations. In particular, aspects described in connection with the system 45 can be combined or can be advantageous in combination with the aspects described above in connection with the combination of the granulator 13 and the dryer 15.

List of reference signs:

| | |
|---|---|
| 1 | Installation |
| 2 | Formulation |
| 3 | Feedstock |
| 4 | Feedstock parameters |
| 5 | Control parameters |
| 5A | Feed conveyor drive control parameters |
| 5B | Granulator drive control parameters |
| 5C | Injection device control parameters |
| 5D | Granulator temperature control device control parameters |
| 5E | Supply air conveyor drive control parameters |
| 5F | Supply air temperature control device control parameters |
| 5G | Exhaust air conveyor drive control parameters |
| 5H | Fluidized bed drive control parameters |
| 6 | actuator |
| 6A | feed conveyor drive |
| 6B | granulator drive |
| 6C | injection device |
| 6D | granulator temperature control device |
| 6E | supply air conveyor drive |
| 6F | supply air temperature control device |
| 6G | exhaust air conveyor drive |
| 6H | fluidized bed drive |
| 7 | state parameter |
| [7] | state parameter value |
| 7A | feed conveyor parameter |
| 7B | granulator parameter |
| 7C | injection device parameter |
| 7D | granulator temperature |
| 7E | supply air conveyor parameter |
| 7F | supply air parameter |
| 7G | exhaust air parameter |
| 7H | fluidized bed parameter |
| 7I | formulation temperature |
| 7J | formulation outlet quantity parameter |
| 7K | formulation property parameter |
| 7L | intermediate product property parameter |
| 8 | sensor |
| 8A | feed conveyor sensor |
| 8B | granulator sensor |
| 8C | injection sensor |
| 8D | granulator temperature sensor(s) |
| 8E | supply air conveyor sensor |
| 8F | supply air sensor |
| 8G | exhaust air conveyor sensor |
| 8H | fluidized bed sensor |
| 8I | formulation temperature sensor |
| 8J | formulation outlet quantity sensor |
| 8K | formulation property sensor |
| 8L | intermediate product sensor |
| 9 | control device |
| 10 | target formulation parameter |
| 10A | database |
| 11 | actual formulation parameter |
| 12 | model |
| 12A | static part |
| 12B | dynamic part |
| 13 | granulator |
| 13A | granulator inlet |
| 13B | granulating liquid |
| 14 | intermediate product |
| 15 | dryer |
| 15A | supply air conveyor |
| 15B | exhaust air conveyor |
| 16 | feed conveyor |
| 16A | storage container |
| 16B | supply device |
| 16C | screw |
| 17 | granulating unit |
| 18 | screw |
| 19 | processing zone |
| 20 | coupling device |
| 21 | intermediate product outlet |
| 22 | housing |
| 23 | housing |
| 24 | intermediate product inlet |
| 25 | fluidized bed |
| 26 | air inlet |
| 27 | air |
| 28 | diffuser |
| 29 | air outlet |
| 30 | separator |
| 31 | formulation outlet |
| 32 | artificial neural network |
| 33 | input layer |
| 34 | hidden layer |
| 35 | output layer |
| 36 | node |
| 37 | edge |
| 38 | weight |
| 39 | reference trajectory |
| 40 | forecast horizon |
| 41 | fixed time window |
| 42 | quality dynamics |
| 43 | planning dimension |
| 44 | process state |
| 45 | system |
| 46 | conveyor installation |
| 47 | components |
| 48 | preparation step |
| 49 | granulating step |
| 50 | drying step |
| 51 | post-processing step |
| 52 | post-processing device |
| 53 | post-processed formulation |
| 54 | final mixture |
| 55 | mixing process |
| 56 | disintegrant and/or binder |
| 57 | tabletting process |
| 58 | administration form |
| t | time |
| tk + p | point in time |
| Δt | sampling time |
| P | past |
| F | future |
| ζ | relative time |
| κ | measurement value |

What is claimed is:

1-15. (canceled)

16. A method for controlling an installation for producing a formulation from a feedstock, wherein the producing comprises a processing of the feedstock with a granulator and drying of an intermediate product, produced from the feedstock with the granulator, by means of a dryer, the method comprising:
based on a model:
(a) using a data processor for determining control parameters of the installation, which are or represent manipulated variables for controlling actuators of the installation, the model considering preset target formulation parameters, which represent desired properties of the formulation produced or to be produced, or
(b) using the data processor for forecasting actual formulation parameters, which represent actual properties of the formulation produced or to be produced, the model taking into account preset or presettable control parameters, which are or represent manipulated variables for controlling actuators of the installation,
wherein the determining and forecasting, by the model, uses state parameters of the installation, which each represent a state of the installation that influences the production, and taking into account, by the model, feedstock parameters, which represent an attribute of the feedstock, and
at least one of:
coupling the dryer to the granulator in such a way that the intermediate product is automatically conveyed without interruption from the granulator into the dryer, and using the model to take into account the continuously running combination of granulation with the granulator and subsequent drying with the dryer, or
determining, by a static part of the model, a base value for the respective control parameter or forecasted actual formulation parameter, and optimizing, by a dynamic part of the model, the base value by means of a forecast.

17. The method according to claim 16, wherein at least one of:
the desired properties of the formulation produced or to be produced are an intended grain property and moisture,
the actual properties of the formulation produced or to be produced are an actual grain property and moisture, or
an attribute of the feedstock is a moisture and/or a grain property.

18. The method according to claim 16, wherein the model or the static part of the model is or comprises an artificial neural network.

19. The method according to claim 18, wherein the artificial neural network comprises input layer nodes in an input layer which correspond to at least one state parameter of the granulator and to at least one state parameter of the dryer.

20. The method according to claim 18, wherein the artificial neural network comprises output layer nodes in an output layer which correspond to the control parameters, while the artificial neural network comprises input layer nodes in the input layer which correspond to the target formulation parameters.

21. The method according to claim 18, wherein the artificial neural network comprises input layer nodes in the input layer which correspond to the control parameters, while the artificial neural network comprises output layer nodes in the output layer which correspond to the forecasted actual formulation parameters.

22. The method according to claim 18, wherein the artificial neural network is or has been trained with different training data sets which each are composed of combinations of the feedstock parameters, state parameters and control parameters as well as actual formulation parameters occurring when these parameters are given.

23. The method according to claim 22, wherein the training data sets each represent a stationary state of the installation, in which the actual formulation parameters and state parameters have assumed an at least substantially static value based on constant control parameters and feedstock parameters.

24. The method according to claim 23, wherein the artificial neural network is or has been trained by subjecting input layer nodes of the artificial neural network to at least one state parameter of the granulator and at least one corresponding state parameter of the dryer of the respective training data set.

25. The method according to claim 24, wherein the input layer nodes are furthermore each subjected to corresponding control parameters or actual formulation parameters insofar as nodes are provided for this purpose in the input layer.

26. The method according to claim 24, wherein values result respectively at output layer nodes of an output layer of the artificial neural network for the respective control parameters or forecasted actual formulation parameters and errors are determined by comparison of these values with the corresponding control parameters or actual formulation parameters of the respective training data set, and wherein the errors are reduced by adjustment of weights of the artificial neural network.

27. The method according to claim 16, wherein the control parameters comprise a control parameter for controlling the granulator as well as at least one control parameter for controlling the dryer.

28. The method according to claim 16, wherein the state parameters comprise at least one parameter which describes an operating state of the granulator and a parameter which describes an operating state of the dryer.

29. The method according to claim 16, further comprising
determining, with the model, the control parameters only on the basis of the feedstock parameters, state parameters and actual formulation parameters.

30. The method according to claim 16, further comprising determining, with the model, the forecasted actual formulation parameters only on the basis of the feedstock parameters, state parameters and control parameters.

31. The method according to claim 16, further comprising taking into account, by the model, long-term effects of changes in the control parameters on the actual formulation parameters and/or state parameters.

32. The method according to claim 16, further comprising forecasting, with the dynamic part of the model, a change in state parameters, and, based on the forecast, adjusting the base values for the control parameters and controlling the installation with the control parameters optimized in this way.

33. A computer-readable storage medium, comprising commands which, when the program is executed by a computer, cause the computer to carry out a method for controlling an installation for producing a formulation from a feedstock, wherein the producing comprises a processing of the feedstock with a granulator and a drying of an intermediate product, produced from the feedstock with the granulator, by means of a dryer, the method comprising:
based on a model:
(a) determining control parameters of the installation, which are or represent manipulated variables for controlling actuators of the installation, the model taking into account preset target formulation parameters, which represent desired properties of the formulation produced or to be produced, or
(b) forecasting actual formulation parameters, which represent actual properties of the formulation produced or to be produced, the model taking into account preset or presettable control parameters, which are or represent manipulated variables for controlling actuators of the installation,
determining and processing, by the model, state parameters of the installation, which each represent a state of the installation which influences the production, and taking into account, by the model, feedstock parameters, which represent an attribute of the feedstock, and
at least one of:
coupling the dryer to the granulator in such a way that the intermediate product is automatically conveyed without interruption from the granulator into the dryer, and the model taking into account the continuously running combination of granulation with the granulator and subsequent drying with the dryer, or
determining, by a static part of the model, a base value for the respective control parameter or forecasted actual formulation parameter, and optimizing, by a dynamic part of the model, the base value by means of a forecast.

34. An installation for producing a formulation from a feedstock, wherein the producing comprises a processing of the feedstock with a granulator and a drying of an intermediate product, produced with the granulator, by means of a dryer, the installation comprising:
sensors for detecting state parameters of the installation, which each represent a state of the installation which influences production,
actuators of the installation for acting directly or indirectly on the feedstock,
a control device for controlling the actuators with control parameters, which are or represent manipulated variables for controlling the actuators, and
a model, on the basis of which the control parameters are determinable and/or actual formulation parameters are forecastable,
wherein target formulation parameters are preset or presettable for the control device, which represent the desired properties of the formulation produced or to be produced,
wherein the granulator is coupled to the dryer in such a way that the intermediate product is automatically conveyed without interruption from the granulator into the dryer, wherein the model is configured to take into account the combination of granulation with the granulator and the continuous subsequent drying with the dryer, and the installation is configured such that the control device determines the control parameters and/or forecasts actual formulation parameters with the model based on the state parameters of the installation; and/or
wherein the model comprises a static part, wherein the control device is configured to determine a base value with the static part for the respective control parameter or actual formulation parameter with the state parameters, and wherein the model comprises a dynamic part, wherein the control device is configured to optimize the base value with the dynamic part by means of a forecast.

35. The installation according to claim 34, further comprising a feedstock forming device for forming the feedstock from a plurality of components and/or a formulation processing device for further processing of the formulation.
